(19) United States
(12) Patent Application Publication
(10) Pub. No.: US 2025/0235521 A1
(43) Pub. Date: Jul. 24, 2025

(54) LIVE MYCOPLASMA GALLISEPTICUM VACCINES

(71) Applicant: UNIVERSITY OF GEORGIA RESEARCH FOUNDATION, INC., Athens, GA (US)

(72) Inventor: Naola M. FERGUSON-NOEL, Loganville, GA (US)

(21) Appl. No.: 18/853,887

(22) PCT Filed: Apr. 5, 2023
(86) PCT No.: PCT/US2023/017527

§ 371 Date: Oct. 3, 2024

Related U.S. Application Data
(60) Provisional application No. 63/327,851, filed on Apr. 6, 2022.
(60) Provisional application No. 63/404,226, filed on Sep. 7, 2022.

(51) Int. Cl. A61K 39/02; A61K 9/00; A61K 39/00; A61P 31/04; C12N 1/20; C12R 1/35

(52) U.S. Cl. CPC A61K 39/0241; A61K 9/0048; A61P 31/04; C12N 1/205; A61K 2039/522; A61K 2039/542; A61K 2039/552; C12R 2001/35

(57) ABSTRACT

The present invention provides Mycoplasma gallisepticum strain K6067 as deposited at the ATCC under Patent Designation PTA-127168, Mycoplasma gallisepticum strain K4110 as deposited at the ATCC under Patent Designation PTA-127282, and progeny and derivatives thereof, for use as a vaccine for the prevention of virulent Mycoplasma gallisepticum infections in the birds of the order Galliformes. Also provided are compositions and methods for administration to birds of the order Galliformes.

### CONTINUING APPLICATION DATA

This application claims the benefit of U.S. Provisional Application Ser. No. 63/327,851, filed Apr. 6, 2022, and U.S. Provisional Application Ser. No. 63/404,226, filed Sep. 7, 2022, each of which are incorporated by reference herein.

### BACKGROUND

Mycoplasma gallisepticum (MG) is an infectious respiratory pathogen of chickens and turkeys. It is the most pathogenic and economically significant Mycoplasma pathogen of poultry. Economic losses from condemnation or downgrading of carcasses, reduced feed and egg production efficiency, and increased medication costs are factors that make this one of the costliest disease problems confronting commercial poultry production worldwide. While different measures such as voluntary monitoring programs, treatment with different antibiotics, and vaccination have been implanted in the poultry industry to control Mycoplasma gallisepticum infection and to decrease economic losses in the poultry industry, MG is still considered an enduring challenge to the commercial poultry industry. Voluntary monitoring programs can be effective, but the value of the long-lived flocks, (breeders and layers) has made the justification to cull and quarantine difficult, especially in areas where MG is widespread and endemic. Increasing legal limitations to antibiotic use in poultry, especially prophylactic in-feed use, as well as increasing reports of resistance to the antibiotics to commonly used to control MG infections decrease the applicability and effectiveness of the option of treatment with different antibiotics. Thus, vaccination becomes an ever more attractive option for MG control. There are currently three commercially available live attenuated vaccines for MG (6/85, F strain and ts-11). All three of these vaccines have pros and cons with respect to safety and efficacy. Thus, there is a need for additional live attenuated vaccines for the control of Mycoplasma gallisepticum infection in poultry.

### SUMMARY OF THE INVENTION

The present invention includes an isolated Mycoplasma gallisepticum strain, wherein the isolated Mycoplasma gallisepticum strain is the K6067 Mycoplasma gallisepticum strain deposited at the ATCC under Patent Designation PTA-127168, or a progeny or derivative thereof. In some aspects, the present invention includes a composition including the isolated Mycoplasma gallisepticum strain. In some aspects, the composition may include water. In some aspects, the composition may include a pharmaceutically acceptable carrier. In some aspects, the composition may include an adjuvant. In some aspects, the composition may be formulated for intranasal, intraocular, oral, mucosal, intramuscular, subcutaneous, or in ovo administration. In some aspects, the composition may be formulated for spraying or aerosolizing.

The present invention includes an essentially biologically pure culture of the K6067 Mycoplasma gallisepticum strain deposited at the ATCC under Patent Designation PTA-127168. In some aspects, the present invention includes a composition including the isolated Mycoplasma gallisepticum strain. In some aspects, the composition may include water. In some aspects, the composition may include a pharmaceutically acceptable carrier. In some aspects, the composition may include an adjuvant. In some aspects, the composition may be formulated for intranasal, intraocular, oral, mucosal, intramuscular, subcutaneous, or in ovo administration. In some aspects, the composition may be formulated for spraying or aerosolizing.

The present invention includes a vaccine including an isolated Mycoplasma gallisepticum strain K6067 deposited at the ATCC under Patent Designation PTA-127168, or a progeny or derivative thereof, as described herein, an essentially biologically pure culture of the K6067 Mycoplasma gallisepticum strain deposited at the ATCC under Patent Designation PTA-127168 as described herein, or a composition as described herein. In some aspects, the vaccine reduces one or more of the clinical signs induced by Mycoplasma gallisepticum infection in poultry. In some aspects, the vaccine reduces the susceptibility of a birds of the order Galliformes to disease induced by Mycoplasma gallisepticum.

The present invention includes a live vaccine for birds of the order Galliformes, the vaccine including an amount of the K6067 Mycoplasma gallisepticum strain deposited at the ATCC under Patent Deposit Designation PTA-127168 or a progeny or derivative thereof, as described herein, sufficient to protect the birds from disease induced by Mycoplasma gallisepticum, and a pharmaceutically acceptable carrier.

The present invention includes an isolated Mycoplasma gallisepticum strain, wherein the isolated Mycoplasma gallisepticum strain is the K4110 Mycoplasma gallisepticum strain deposited at the ATCC under Patent Designation PTA-127282, or a progeny or derivative thereof. In some aspects, the present invention includes a composition including the isolated Mycoplasma gallisepticum strain. In some aspects, the composition may include water. In some aspects, the composition may include a pharmaceutically acceptable carrier. In some aspects, the composition may include an adjuvant. In some aspects, the composition may be formulated for intranasal, intraocular, oral, mucosal, intramuscular, subcutaneous, or in ovo administration. In some aspects, the composition may be formulated for spraying or aerosolizing.

The present invention includes an essentially biologically pure culture of the K4110 Mycoplasma gallisepticum strain deposited at the ATCC under Patent Designation PTA-127282. In some aspects, the present invention includes a composition including the isolated Mycoplasma gallisepticum strain. In some aspects, the composition may include water. In some aspects, the composition may include a pharmaceutically acceptable carrier. In some aspects, the composition may include an adjuvant. In some aspects, the composition may be formulated for intranasal, intraocular, oral, mucosal, intramuscular, subcutaneous, or in ovo administration. In some aspects, the composition may be formulated for spraying or aerosolizing.

The present invention includes a vaccine including an isolated Mycoplasma gallisepticum strain K4110 deposited at the ATCC under Patent Designation PTA-127282, or a progeny or derivative thereof, as described herein, an essentially biologically pure culture of the K4110 Mycoplasma gallisepticum strain deposited at the ATCC under Patent Designation PTA-127282 as described herein, or a composition as described herein. In some aspects, the vaccine reduces one or more of the clinical signs induced by Mycoplasma gallisepticum infection in poultry. In some aspects, the vaccine reduces the susceptibility of a birds of the order Galliformes to disease induced by Mycoplasma gallisepticum.

The present invention includes a live vaccine for birds of the order Galliformes, the vaccine including an amount of the K4110 Mycoplasma gallisepticum strain deposited at the ATCC under Patent Deposit Designation PTA-127282 or a progeny or derivative thereof, as described herein, sufficient to protect the birds from disease induced by Mycoplasma gallisepticum, and a pharmaceutically acceptable carrier.

In some aspects, an isolated Mycoplasma gallisepticum strain as described herein, a composition as described herein, o a vaccine as described herein may be lyophilized, freeze dried, frozen, or an effervescent tablet.

The present invention includes a kit including an isolated Mycoplasma gallisepticum strain as described herein, a composition as described herein, or a vaccine as described herein and printed instructions, wherein the contents of the kit are contained within packaging material.

The present invention includes a method of producing an immune response to Mycoplasma gallisepticum in a bird, the method including administering an isolated Mycoplasma gallisepticum strain as described herein, a composition as described herein, or a vaccine as described herein. In some aspects, administration is intranasal, intraocular, oral, mucosal, intramuscular, subcutaneous, or in ovo. In some aspects, administration is by eye drop, by aerosol, or by drinking water. In some aspects, the bird is of the order Galliformes. In some aspects, the bird is a chicken or a turkey.

The present invention includes a method for reducing susceptibility of a bird against disease induced by Mycoplasma gallisepticum, the method including administering an isolated Mycoplasma gallisepticum strain as described herein, a composition as described herein, or a vaccine as described herein to the bird. In some aspects, administration is intranasal, intraocular, oral, mucosal, intramuscular, subcutaneous, or in ovo. In some aspects, administration is by eye drop, by aerosol, or by drinking water. In some aspects, the bird is of the order Galliformes. In some aspects, the bird is a chicken or a turkey.

The present invention includes a method for protecting a bird against Mycoplasma gallisepticum infection, the method including administering an isolated Mycoplasma gallisepticum strain as described herein, a composition as described herein, or a vaccine as described herein to the bird. In some aspects, administration is intranasal, intraocular, oral, mucosal, intramuscular, subcutaneous, or in ovo. In some aspects, administration is by eye drop, by aerosol, or by drinking water. In some aspects, the bird is of the order Galliformes. In some aspects, the bird is a chicken or a turkey.

The present invention includes a method of reducing one or more clinical signs induced by a Mycoplasma gallisepticum infection in a bird, the method including administering an effective amount of an isolated Mycoplasma gallisepticum strain as described herein, a composition as described herein, or a vaccine as described herein to the bird. In some aspects, administration is intranasal, intraocular, oral, mucosal, intramuscular, subcutaneous, or in ovo. In some aspects, administration is by eye drop, by aerosol, or by drinking water. In some aspects, the bird is of the order Galliformes. In some aspects, the bird is a chicken or a turkey.

As used herein, “isolated” refers to material removed from its original environment (e.g., the natural environment if it is naturally occurring), and thus is altered “by the hand of man” from its natural state.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

In several places throughout the application, guidance is provided through lists of examples, which examples can be used in various combinations. In each instance, the recited list serves only as a representative group and should not be interpreted as an exclusive list. It is to be understood that the particular examples, materials, amounts, and procedures are to be interpreted broadly in accordance with the scope and spirit of the invention as set forth herein.

All headings throughout are for the convenience of the reader and should not be used to limit the meaning of the text that follows the heading, unless so specified.

### DETAILED DESCRIPTION

The bacteria Mycoplasma gallisepticum is a member of the Mycoplasma genus. It is the causative agent of chronic respiratory disease in chickens and infectious sinusitis in turkeys, chickens, game birds, pigeons, and passerine birds of all ages. It is found throughout the world and infection may be referred to as infectious synovitis, avian mycoplasmosis, infectious sinusitis, or Mycoplasma arthritis. M gallisepticum is transmitted vertically within some eggs (transovarian) from infected breeders to progeny, and horizontally via infectious aerosols and through contamination of feed, water, and the environment, and by human activity on fomites (shoes, equipment, etc). It is of economic importance because infection can cause a drop in egg production (El-Gazzar, “Mycoplasma gallisepticum Infection in Poultry,” Merk Veterinary Manual, 2020).

The present invention provides Mycoplasma gallisepticum (MG) strain K6067 and progeny and derivatives thereof that are immunogenic and stable when administered as live formulations. The present invention also provides Mycoplasma gallisepticum (MG) strain K4110 and progeny and derivatives thereof that are immunogenic and stable when administered as live formulations. Formulations of Mycoplasma gallisepticum of the present invention are safe and efficacious to inhibit Mycoplasma gallisepticum infections and will be useful in reducing the incidence and severity of disease of Mycoplasma gallisepticum infections in birds.

Mycoplasma gallisepticum strain K6707 was deposited with the American Type Culture Collection (ATCC©), 10801 University Boulevard, Manassas, VA 20110-2209, USA, as PTA-127168 on Nov. 24, 2021. This strain was deposited in accordance with the Budapest Treaty on the International Recognition of the Deposit of Microorganisms for the Purposes of Patent Procedure. Mycoplasma gallisepticum strain K6067 as deposited with the ATCC© as PTA-127168 is also referred to herein as Mycoplasma gallisepticum strain 6067, M gallisepticum strain 6067, MG strain K6067, K6067, MG strain K6067 ATCC PTA-127168, K6067 ATCC PTA-127168, MG strain K6067 PTA-127168, K6067 PTA-127168, and ATCC© PTA-127168.

Mycoplasma gallisepticum strain K4110 was deposited with the American Type Culture Collection (ATCC©), 10801 University Boulevard, Manassas, VA 20110-2209, USA, as PTA-127282 on Jul. 20, 2022. This strain was deposited in accordance with the Budapest Treaty on the International Recognition of the Deposit of Microorganisms for the Purposes of Patent Procedure. Mycoplasma gallisepticum strain K4110 as deposited with the ATCC© as PTA-127282 is also referred to herein as Mycoplasma gallisepticum strain 4110, M. gallisepticum strain 4110, MG strain K4110, K4110, MG strain K4110 ATCC PTA-127282, K4110 ATCC PTA-127282, MG strain K4110 PTA-127282, K4110 PTA-127282, and ATCC® PTA-127282.

The present invention includes isolated Mycoplasma gallisepticum (MG) strain K6067 with the ATCC Patent Deposit Designation PTA-127168. As used herein, “isolated” refers to material removed from its original environment (e.g., the natural environment if it is naturally occurring), and thus is altered “by the hand of man” from its natural state. Also included are biologically pure cultures of Mycoplasma gallisepticum (MG) strain K6067 with the ATCC Patent Deposit Designation PTA-127168.

The present invention includes isolated Mycoplasma gallisepticum (MG) strain K4110 with the ATCC Patent Deposit Designation PTA-127282. Also included are biologically pure cultures of Mycoplasma gallisepticum (MG) strain K4110 with the ATCC Patent Deposit Designation PTA-127282.

Also included in the present invention are isolated progeny and isolated derivatives of Mycoplasma gallisepticum strain K6067 deposited with the ATCC Patent Deposit Designation as PTA-127168 with equivalent or similar biological, serological, and/or genetic characteristics. As used herein, serological, biological, and genetic characteristics may include one or more of the characteristics described in the data in the Examples included herewith. More particularly, progeny or derivative of the K6067 strain deposited with the ATCC as PTA-127168 may retain the particularly favorable protective properties belonging to the present invention. Progeny or derivatives of Mycoplasma gallisepticum strain K6067 deposited with the ATCC as PTA-127168 may be obtained by any of the various methods for propagating Mycoplasma gallisepticum known in the art, including, but not limited to, for example, in vitro culture or back passage in a bird. Derivatives of Mycoplasma gallisepticum strain K6067 ATCC PTA-127168 may include genetically modified versions of the deposited MG K6067 strain. Such manipulations include, but are not limited to, mutagenizing the MG strain, or introducing genes or gene cassettes encoding alternative proteins or nonfunctional proteins, or noncoding nucleotide sequences into the MG organism.

Also included in the present invention are isolated progeny and isolated derivatives of Mycoplasma gallisepticum strain K4110 deposited with the ATCC Patent Deposit Designation as PTA-127282 with equivalent or similar biological, serological, and/or genetic characteristics. As used herein, serological, biological, and genetic characteristics may include one or more of the characteristics described in the data in the Examples included herewith. More particularly, progeny or derivative of the K4110 strain deposited with the ATCC as PTA-127282 may retain the particularly favorable protective properties belonging to the present invention. Progeny or derivatives of Mycoplasma gallisepticum strain K4110 deposited with the ATCC as PTA-127282 may be obtained by any of the various methods for propagating Mycoplasma gallisepticum known in the art, including, but not limited to, for example, in vitro culture or back passage in a bird. Derivatives of Mycoplasma gallisepticum strain K411 ATCC PTA-127282 may include genetically modified versions of the deposited MG K4110 strain. Such manipulations include, but are not limited to, mutagenizing the MG strain, or introducing genes or gene cassettes encoding alternative proteins or nonfunctional proteins, or noncoding nucleotide sequences into the MG organism.

A Mycoplasma gallisepticum (MG) strain K6067 isolate, a MG strain K4110, and progeny and derivatives thereof, as described herein may be propagated by conventional methods, including, but not limited to, any of those described in the examples section included herewith. For example, a Mycoplasma gallisepticum strain of the present invention may be cultured with Frey's modified broth and agar (Ferguson-Noel and Kleven, “Mycoplasma species,” In: Williams S M, Dufour-Zavala L, Jackwood M W, Lee M D, Lupiani B, Reed W M, Spackman E. Woolcock P R, editors. A Laboratory Manual for the Isolation, Identification and Characterization of Avian Pathogens. American Association of Avian Pathologists. p. 63-70; 2016).

MG strain K6067, MG strain K4110, and progeny and derivatives thereof, may be identified and differentiated from other Mycoplasma spp. and other M. gallisepticum strains using any of many known techniques, including, for example, direct immunofluorescence with species-specific antibodies, serum plate agglutination (SPA) test, hemagglutination inhibition test (HI), enzyme-linked immunosorbent assay (ELISA), and real-time quantitative PCR (qPCR) (Raviv and Kleven, 2009, Avian Dis; 53:103-107), whole genome sequencing, and targeted DNA sequencing of the 16S-23S rRNA intergenic space region (IGSR) (Papazisi et al., 2003, Microbiology; 149:2307-16) and mgc2 cytadhesin (Hnatow et al., 1998, Infect Immun; 66:3436-42).

Mycoplasma gallisepticum (MG) strain K6067, Mycoplasma gallisepticum (MG) strain K4110, and progeny and derivatives thereof as described herein may be administered as a live attenuated MG vaccine to poultry, including chickens and turkeys. While different measures such as voluntary monitoring programs, vaccination and treatment with different antibiotics have been implanted in the poultry industry to control Mycoplasma gallisepticum (MG) infection and to decrease economic losses in the poultry industry, MG is still considered an enduring challenge to the commercial poultry industry. Voluntary monitoring programs can be effective, but the value of the long-lived flocks, (breeders and layers), has made the justification to cull and quarantine difficult, especially in areas where MG is widespread and endemic. Further, increasing legal limitations to antibiotic use in poultry (especially prophylactic in-feed use) in Europe and the US (Veterinary Feed Directive Journal. 80 number 106, 2015) as well as increasing reports of resistance to the antibiotics to commonly used to control MG infections (de Jong et al., 2021, Avian Pathology: 50(2):161-173) decrease the applicability and effectiveness of this option. In these situations, vaccination becomes an ever more attractive option for MG control.

Currently available, immunizing agents include bacterins, live attenuated and recombinant vaccines. There are some advantages with bacterins as they are not infectious or transmissible and cannot revert to virulence (Sasipreeyajan et al., 1987, Avian Dis; 31:776-81); bacterins have been shown to reduce egg production losses, ovarian regression, and egg transmission of MG (Glisson and Kleven, 1985, Avian Dis; 29:408-15). However, bacterins provide very limited protection against infection, tracheal and air sac lesions (Talkington and Kleven, 1985, Avian Dis; 29:998-1003). The labor cost and individual handling of the birds for intramuscular injection may cause stress and there is also a rick of local vaccine reactions. For MG recombinant vaccines, a fowl pox-vectored MG vaccine is commercially available but the efficacy against respiratory lesions caused by pathogenic MG strain was reported to be significantly less than bacterins and live attenuated vaccines (Ferguson-Noel et al., 2012, Avian Dis; 56:272-275). Recently, an adenovirus-vectored MG vaccine based on the TM-1 protein was developed but further study is needed to investigate the efficacy (Dongchao Zhang et al., 2018, Avian Pathology; 42(2):213-222). There are several genes and virulence factors (Yu et al., 2019, Infection and Immunity; 87:e00248-19; Papazisi et al., 2002, Infect Immun; 70:6839-45; and Indikova et al., 2014, Vet Res; 45:99) that play different roles in MG pathogenicity and immunity, choosing one specific gene to produce adequately effective and protective MG vaccine may be challenging.

There are currently three commercially available live attenuated vaccines for MG as 6/85, F strain and ts-11. These vaccines all have pros and cons with respect to safety and efficacy; the above—an ideal MG vaccine should be immunogenic and at the same time avirulent while still be affordable and able to prevent colonization of the pathogenic MG strain after exposure (Cummings and Kleven, 1986, Avian Dis; 30:169-71). The 6/85 vaccine is very safe (Evans and Hafez, 1992, Avian Dis; 36:197-201; Ley et al., 1997, Avian Dis; 41:187-94; and Zaki et al., 2004, Avian Dis; 48:642-6), however, it was reported that the vaccine is not highly efficacious and colonizes weakly (Evans and Hafez, 1992, Avian Dis; 36:197-201; and Kleven et al., 1998, Avian Dis; 42:300-6). Additionally, poor serological response might pose problems in evaluating the immunization status of vaccinated flocks with serological assays (Throne Steinlage et al., 2003, Avian Dis; 47:499-505).

F-strain was the first live vaccine introduced to the poultry industry to control MG infections (Yamamoto and Adler, 1958, J Infect Dis; 102:143-52) and it is currently the least attenuated live MG vaccine commercially available. Due to reports of being pathogenic in turkeys (Lin and Kleven, 1982, Avian Dis; 26:360-4), being transmissible to the non-vaccinated birds (Kleven, 1981, Avian Dis; 25:1005-18), and the risk of increased virulence when it circulates and backpassages in a flock (Gharaibeh et al., 2011, Avian Dis; 55:212-6) the use of F-strain is not allowed in some areas (such as Minnesota, USA, Israel, and Japan). However, F-strain is very effective for displacement of the pathogenic MG strains (Kleven et al., 1990, Avian Dis; 34:984-90) as well as reducing air sac and tracheal lesions (Kleven, 1986, Avian Diseases; 30 No. 1:169-171) and decreasing virulent MG egg transmission (Glisson and Kleven, 1984, Avian Dis; 28:406-15). The other live MG vaccine that persists in the trachea of the vaccinated for the life of the flock similar to F strain (Kleven, 1998, Poult Sci; 77:1146-9) is ts-11 (Whithear et al., 1990, Aust Vet J; 67:168-74). This vaccine efficacious in preventing respiratory lesions (Abd-el-Motelib and Kleven, 1993, Avian Dis; 37:981-7), egg production losses (Barbour et al., 2000, Poult Sci; 79:1730-5) and ovarian regression (Whithear et al., 1990, IOM Lett; 1:361-362). Although ts-11 is safe to use in turkeys, it is unable to colonize the trachea well enough to elicit a protective immunity (Whithear et al., 1990, Aust Vet J; 67:159-65; and Wijesurendra et al., 2017, Avian Pathol; 1-10; 2017). Reversion to virulence of ts-11 has also been reported (Armour et al., 2015, Avian Pathol; 44:296-304). Recently, ts-304, a GapA+ ts-11 vaccine, has been developed. While the persistence of the vaccine is still similar to ts-11 (Condello et al., 2020, Veterinary Microbiology; 251:108883), it is reported to be safe and efficacious to use in turkeys (Kanci et al., 2018, Vaccine; 36(18):2487-2493).

The present invention includes compositions and vaccines of the M. gallisepticum isolates and progeny and derivatives thereof as described herein. In preferred embodiments, the M. gallisepticum isolate is live. In some embodiments, the M. gallisepticum isolate may be inactivated or killed. A M. gallisepticum strain and compositions and vaccines thereof of the present invention may be stored until use in any of a variety of forms. For example, such materials, may be lyophilized or freeze dried and may be rehydrated for use. In some embodiments, a M. gallisepticum strain or composition or vaccine thereof may be frozen.

In some embodiments, a M. gallisepticum isolate or composition or vaccine thereof may be formulated as an effervescent table. Such effervescent tablets may, for example, be packaged in lightweight aluminum blisters. The table may be dissolved in water and administered, for example, orally, nasally, or by aerosol spray, whereby droplets enter via the mucus membranes of the birds.

Compositions and vaccines of the present invention may include, for example, water or culture medium. Such compositions and vaccines may include one or more suitable pharmaceutically acceptable carriers, stabilizers, preservatives, diluents, and/or buffers. Suitable stabilizers include, for example, SPGA, carbohydrates (such as sorbitol, mannitol, starch, sucrose, dextrin, or glucose), or proteins (such as albumin or casein). A stabilizer is particularly advantageous when a dry vaccine preparation is prepared by lyophilization. Suitable preservatives include, for example, thimerosal, merthiolate, and gentamicin. Diluents include, but are not limited to, water, aqueous buffer (such as buffered saline), alcohols, and polyols (such as glycerol).

A composition or vaccine of the present invention may also include one or more compounds with adjuvant activity. Suitable compounds or compositions for this purpose include aluminum hydroxide, aluminum phosphate, aluminum oxide, plant oils, animal oils, oil-in-water or water-in-oil emulsion based on, for example a mineral oil, such as Bayol F™ or Marcol 52™ Complete Freund's adjuvant, incomplete Freund's adjuvant, or a vegetable oil such as vitamin E acetate, and saponins.

A composition or vaccine of the present invention may further include one or more immunogens derived from other pathogens infectious to poultry. Such immunogens may be derived from, for example, Mycoplasma synoviae (MS), Marek's disease virus (MDV), infectious bronchitis virus (IBV), Newcastle disease virus (NDV), egg drop syndrome (EDS) virus, turkey rhinotracheitis virus (TRTV), poxvirus, reovirus, chicken parvovirus, and avian nephritis virus (including, but not limited to ANV-1 and ANV-2).

Compositions and vaccines of the present invention may be substantially pure. As used herein, “substantially pure” will mean material essentially free of macromolecules or other biological entities that would normally be found with it in nature.

Compositions and vaccines of the present invention may be administered to birds of any of a variety of avian species that are susceptible to Mycoplasma gallisepticum infection, including, but not limited to, poultry, birds of the order Galliformes, and exotic bird species. Birds of the order Galliformes include, but are not limited to, chickens, turkeys, grouse, quails, and pheasants. As used herein, poultry includes domesticated birds that are kept for the purpose of collecting their eggs or killing for their meat and/or feathers. These most typically are members of the superorder Galloanserae (fowl), especially the order Galliformes (which includes, for example, chickens, quail, turkeys, and grouse) and the family Anatidae (in order Anseriformes), commonly known as “waterfowl” (including, for example, ducks, geese, and swans). Poultry may also include other birds which are killed for their meat, such as pigeons or doves or birds considered to be game, like pheasants. Chickens include, but are not limited to, hens, roosters, broilers, roasters, layers, breeders, the offspring of breeder hens, and layers. As used herein, the term “susceptible to” means the possibility or actuality of a detrimental response to the referenced microorganism, such as, for example, reduced vigor or a failure to thrive, when compared to a non-susceptible individuals or groups, and/or one or more pathological state(s) indicative of Mycoplasma gallisepticum infection.

Compositions and vaccines of the present invention may be formulated for delivery by any of a variety of routes known in the veterinary arts, including, but not limited to, for example, mucosal, intranasal, intraocular, or oral administration. Compositions and vaccines of the present invention may be formulated for delivery to the respiratory mucosa and may be administered such that it is immediately or eventually brought into contact with the bird's respiratory mucosal membranes. A composition or vaccine of the present invention may be administered by any suitable known method of inoculating poultry including, but not limited to, nasally, ocularly, by eye drop, by injection, in drinking water, in the feed, in ovo, maternally, by respiratory inhalation, and the like. When administered by injection, the immunogenic composition or vaccine may be administered parenterally. Parenteral administration includes, for example, administration by intravenous, subcutaneous, intramuscular, or intraperitoneal injection.

A composition or vaccine may be formulated for administered by mass administration techniques such as by placing the vaccine in drinking water or by spraying or aerosolizing. A composition may be administered by spraying an individual or the flock with a solution, such aerosol delivery may involve the administration of the composition incorporated in small liquid particles. Such spray-type particles may have a droplet size ranging from between about 10 to about 100 microns, more preferably, a droplet size from between about <1 to about 50 microns. For the generation of the small particles, conventional spray-apparatus and aerosol generators may be used, such as the commercially available spray generators for knapsack spray, hatchery spray and atomist spray. Administration through drinking water may can be carried out using conventional apparatus.

A composition or vaccine of the present invention may be administered to poultry before or after hatching. In ovo vaccination may take place, for example, at about 13 days, about 14 days, about 15 days, about 16 days, about 17 days, about 18 days, about 19 days, about 20 days, or at any range thereof. For in ovo delivery, laying stock or reproduction stock may be vaccinated, for example, at about 6-12 weeks of age and boosted at about 16-20 weeks of age. Such laying stock or reproduction stock may be vaccinated at about 6, at about 7, at about 8, at about 9, at about 10, at about 11, or at about 12 weeks of age. Also, in some embodiments, such laying stock or reproduction stock may be vaccinated within about the first two weeks of age. Such laying stock or reproduction stock may be boosted at about 16, at about 17, at about 18, at about 19, or at about 20 weeks of age. The offspring of such laying stock or reproduction stock may demonstrate an antibody titer to Mycoplasma gallisepticum, which may prevent or mitigate the symptoms of a Mycoplasma gallisepticum infection in the offspring

Poultry may receive a composition or vaccine as described herein at a variety of ages. With delivery after hatching, materials may be delivered at any suitable age, including, but not limited to, about one to three days old, about one week after hatching, about two weeks after hatching, about three weeks after hatching, about four weeks after hatching, about five weeks after hatching, about six weeks after hatching, or any range thereof. The chickens may be vaccinated only once. Or, if two doses of vaccine are used, the first is given, for example, when the chickens are 3 days to a week old and subsequently after a further 1-10 weeks.

Multiple doses of the composition can be administered throughout the life of the chicken. As maternal immunity is a primary source of providing protection to broiler progeny, breeder chickens are typically vaccinated, although broiler chickens can be vaccinated if so desired.

Compositions and vaccines of the present invention may be adjusted to include a designated concentration of Mycoplasma gallisepticum. Organisms may be measured as color changing units. Color changing units, also referred to herein as “ccu,” of Mycoplasma gallisepticum can be quantified using established standard methodology, including, for example, protocols set forth in Rodwell and Whitcomb (In “Methods in Mycoplasmology,” Eds. Razin and Tully, 1993). For example, compositions or vaccines may have concentration of about 50, about 100, about 1×10² ccu/ml, about 2.5×10² ccu/ml, about 5×10² ccu/ml, about 1×10³ ccu/ml, about 2.5×10³ ccu/ml, about 5×10³ ceu/ml, about 1×10⁴ ceu/ml, about 2.5×10⁴ ceu/ml, about 5×10⁴ ccu/ml, about 1×10⁵ ccu/ml, about 2.5×10⁵ ccu/ml, about 5×10⁵ ceu/ml, about 1×10⁶ ceu/ml, about 2.5×10⁶ ccu/ml, about 5×10⁶ ccu/ml, about 1×10⁷ ccu/ml, about 2.5×10⁷ ccu/ml, about 5×10⁷ ceu/ml, 1×10⁸ ceu/ml, about 2.5×10⁸ ceu/ml, about 5×10⁸ ceu/ml, about 1×10⁹ ccu/ml, about 2.5×10⁹ ccu/ml, or about 5×10⁹ ccu/ml, and any range thereof (such as, for example, about 1×10⁵ ccu/ml to about 1×10⁶ ccu/ml) may be used. In some applications, an effective amount may be administered to a single bird at one drop per eye per bird. One drop may be approximately 0.05 ml to 0.1 ml.

Mycoplasma gallisepticum strains of the present invention may be administered to birds to reduce susceptibility to Mycoplasma gallisepticum infection. With such administration, the materials do not result in significant clinical signs or lesions indicative of Mycoplasma gallisepticum. Accordingly, it is an object of the present invention to provide immunological materials that with administration do not result in significant clinical signs or lesions indicative of MS disease. It is another object to provide immunological materials of low virulence.

The present invention includes a method of producing an anti-MG immune response in poultry, the method including administering a Mycoplasma gallisepticum strain, composition, or vaccine as described herein. In some aspects, immunity includes humoral and/or cellular immunity. With a humoral response, anti-MS antibodies may be measured, for example, by the serum plate agglutination (SPA) test (using for example, commercial antigen (Charles River Laboratories International, Inc., Wilmington, MA)); the hemagglutination inhibition (HI) test; and the enzyme-linked immunosorbent assay (ELISA) test (using for example, a commercial kit (IDEXX, Westbrook, Maine)). The SPA and HI test procedures are described in more detail in Ferguson-Noel et al. (Ferguson-Noel, N., and S. H. Kleven Mycoplasma species. In: A Laboratory Manual for the Isolation, Identification and Characterization of Avian Pathogens, Sixth ed. S. M. Williams, L. Dufour-Zavala, M. W. Jackwood, M. D. Lee, B. Lupiani, W. M. Reed, E. Spackman and P. R. Woolcock, eds. American Association of Avian Pathologists. pp 63-70. 2016). In some aspects, immunity includes mucosal immunity.

The present invention includes a method of reducing, inhibiting, or preventing an MG infection in poultry, the method including administering an isolated Mycoplasma gallisepticum strain, composition or vaccine as described herein. Administration of an isolated Mycoplasma gallisepticum strain, composition, or vaccine as described herein may result in the reduction, inhibition, or prevention of one or more of the disease manifestations of challenge with a further infection with MG, including one or more of the disease manifestations of infectious MG. Such clinical signs and symptoms may include, for example, one or more of respiratory distress, tracheal rales, difficulty breathing, coughing, sneezing, nasal discharge, swollen eyelids, ocular discharge, impaired vision, conjunctivitis, frothiness about the eyes, swelling of the infraorbital sinuses, sinusitis, catarrhal sinusitis, tracheitis, airsacculitis, pneumonia, leg problems, stunting, depression, weight loss, inappetence, slow growth, reduced hatchability, reduced chick viability, abnormal feathers, poor productivity, reduction in feed efficiency, reduction in weight gain, drop in egg production, condemnations at processing and/or increased mortality.

The invention also provides a kit including Mycoplasma gallisepticum strain K6067 and/or a progeny or derivative thereof as described herein. The kit may include one or more containers filled with a Mycoplasma gallisepticum of the present invention. The Mycoplasma gallisepticum strain K6067 may be lyophilized.

The invention also provides a kit including Mycoplasma gallisepticum strain K4110 and/or a progeny or derivative thereof as described herein. The kit may include one or more containers filled with a Mycoplasma gallisepticum of the present invention. The Mycoplasma gallisepticum strain K4110 may be lyophilized.

A kit may include additional, separate containers of other strains of Mycoplasma gallisepticum or other pathogens of poultry. Additionally, the kit may include other reagents such as buffers and solutions needed to practice the invention are also included. Optionally associated with such container(s) can be a notice or printed instructions. A kit of the present invention may include “packaging material.” As used herein, the term “packaging material” refers to one or more physical structures used to house the contents of the kit. Packaging material is constructed by well-known methods, preferably to provide a sterile, contaminant-free environment. Packaging material may be a solid matrix or a material such as glass, plastic, paper, foil, and the like. Thus, for example, a package can be a glass or plastic vial used to contain ccu quantities of Mycoplasma gallisepticum strain K6067 or Mycoplasma gallisepticum strain K4110.

Exemplary Embodiments of the present invention include, but are not limited to, the following.
1. An isolated Mycoplasma gallisepticum strain, wherein the isolated Mycoplasma gallisepticum strain is the K6067 Mycoplasma gallisepticum strain deposited at the ATCC under Patent Designation PTA-127168, or a progeny or derivative thereof.
2. An essentially biologically pure culture of the K6067 Mycoplasma gallisepticum strain deposited at the ATCC under Patent Designation PTA-127168.
3. An isolated Mycoplasma gallisepticum strain, wherein the isolated Mycoplasma gallisepticum strain is the K4110 Mycoplasma gallisepticum strain deposited at the ATCC under Patent Designation PTA-127282, or a progeny or derivative thereof.
4. An essentially biologically pure culture of the K4110 Mycoplasma gallisepticum strain deposited at the ATCC under Patent Designation PTA-127282.
5. A composition comprising the isolated Mycoplasma gallisepticum of any one of Embodiments 1 to 4.
6. The composition of Embodiment 5 comprising water.
7. The composition of Embodiment 5 comprising a pharmaceutically acceptable carrier.
8. The composition of any one of Embodiments 5 to 7 comprising an adjuvant.
9. The composition of any one of Embodiments 5 to 8, wherein the composition is formulated for intranasal, intraocular, oral, mucosal, intramuscular, subcutaneous, or in ovo administration.
10. The composition of any one of Embodiments 5 to 9, wherein the composition is formulated for spraying or aerosolizing.
11. A vaccine comprising the isolated Mycoplasma gallisepticum of any one of Embodiments 1 to 4 or the composition of any one of Embodiments 5 to 10.
12. The vaccine of Embodiment 11, wherein the vaccine reduces one or more of the clinical signs induced by Mycoplasma gallisepticum infection in poultry.
13. The vaccine of Embodiment 11 or 12, wherein the vaccine reduces the susceptibility of a birds of the order Galliformes to disease induced by Mycoplasma gallisepticum.
14. A live vaccine for birds of the order Galliformes, the vaccine comprising an amount of the K6067 Mycoplasma gallisepticum strain deposited at the ATCC under Patent Deposit Designation PTA-127168 or a progeny or derivative thereof, sufficient to protect the birds from disease induced by Mycoplasma gallisepticum, and a pharmaceutically acceptable carrier.
15. A live vaccine for birds of the order Galliformes, the vaccine comprising an amount of the K4110 Mycoplasma gallisepticum strain deposited at the ATCC under Patent Deposit Designation PTA-127282 or a progeny or derivative thereof, sufficient to protect the birds from disease induced by Mycoplasma gallisepticum, and a pharmaceutically acceptable carrier.
16. The isolated Mycoplasma gallisepticum, the composition, or the vaccine of any one of Embodiments 1 to 15, wherein the isolated Mycoplasma gallisepticum, composition, or vaccine is lyophilized, freeze dried, frozen, or an effervescent tablet.
17. A kit comprising the isolated Mycoplasma gallisepticum, the composition, or the vaccine of any one of Embodiments 1 to 16 and printed instructions, wherein the contents of the kit are contained within packaging material.
18. An effervescent tablet comprising the isolated Mycoplasma gallisepticum, the composition, or the vaccine of any one of Embodiments 1 to 16.
19. A method of producing an immune response to Mycoplasma gallisepticum in a bird, the method comprising administering the isolated Mycoplasma gallisepticum, the composition, or the vaccine of any one of Embodiments 1 to 16 to the bird.
20. A method for reducing susceptibility of a bird against disease induced by Mycoplasma gallisepticum, the method comprising administering the isolated Mycoplasma gallisepticum, the composition, or the vaccine of any one of Embodiments 1 to 16 to the bird.
21. A method for protecting a bird against Mycoplasma gallisepticum infection, the method comprising administering the isolated Mycoplasma gallisepticum, the composition, or the vaccine of any one of Embodiments 1 to 16 to the bird.
22. A method of reducing one or more clinical signs induced by a Mycoplasma gallisepticum infection in a bird, the method comprising administering an effective amount of the isolated Mycoplasma gallisepticum, the composition, or the vaccine of any one of Embodiments 1 to 16 to the bird.
23. The method of any one of Embodiments 19 to 22, wherein administration is intranasal, intraocular, oral, mucosal, intramuscular, subcutaneous, or in ovo.
24. The method of any one of Embodiments 19 to 22, wherein administration is by eye drop, by aerosol, or by drinking water.
25. The method of any one of Embodiments 19 to 24, wherein the bird comprises a bird of the order Galliformes.
26. The method of any one of Embodiment 19 to 25, wherein the bird comprises a chicken or a turkey.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Evaluation of Mycoplasma gallisepticum Strains as Live Vaccines in Chickens and Turkeys

Ten Mycoplasma gallisepticum (MG) isolates were selected based on case history and analysis of targeted sequencing results from the isolate reservoir at Poultry Diagnostic and Research Center (PDRC). The safety of the isolates was evaluated in turkeys and two isolates (K4110A and K6067) was selected based on successful colonization of the trachea while showing no clinical signs or gross lesions at the necropsy (Trials 1 and 2). In Trial 3 the safety and efficacy of both isolates were evaluated in chickens; SPF chickens were vaccinated via eyedrop at 4 weeks of age and R strain was used to challenge the chickens via aerosol 4 weeks post vaccination. Both groups inoculated the vaccine candidates did not show significant differences in lesions compared to the negative controls indicating that the candidates are safe in chickens. Both vaccinated groups also had significantly lower lesions post challenge indicating that there are efficacious vaccines. Further investigation was performed to determine the most protective route of administration and dose required for each candidate. The minimum protective doses 50% for both K6067 and K4110A was 10⁴-10⁵ ccu/mL via the eye drop route of administration (Trials 4 and 5). No vertical transmission was detected in Trial 6 In conclusion and according to the results of the studies, both K6067 and K4110A have potential live vaccine candidates as these initial studies indicate that they are safe in turkeys and chickens as well efficacious.

### Materials and Methods

### MG Strains and Isolates

Ten vaccine candidates (Table 1) were selected from MG culture repository at Poultry Diagnostic and Research Center (University of Georgia, Athens, GA) based on criteria including indication that the naturally attenuated (no case history clinical signs or mortality), as well as rapid and consistent in vitro growth rate. Targeted DNA sequencing results of the 16S-23S rRNA intergenic space region (IGSR) (Papazisi et al., 2003, Microbiology; 149:2307-16) and mgc2 cytadhesin (Hnatow et al., 1998, Infect Immun; 66:3436-42) gene were compared to the PDRC MG sequence database to avoid isolates that were highly similar to commercially available vaccines (Ferguson et al., 2005, Microbiology; 151:1883-1893). The analysis and comparison of the sequences were performed with NCBI Blast (Johnson et al., 2008, Nucleic Acids Research; 36 (suppl 2):W5-W9) and MegAlign Software (Lasergene package, DNAStar, Inc., Madison, WI). Whole genome sequencing in both low (˜10p) and high (˜50p) passages of the candidates to evaluate the genetic stability following in vitro passage and to compare whole genome sequences to MG genome sequences available either publicly or in the whole genome library of Mycoplasma laboratory at PDRC. For isolates selected for whole genome analysis, DNA was extracted following growth in Frey's modified broth; the cells were centrifuged at 13,000×g for 3 minutes, supernatant discarded, and the cell pellets were reconstituted in 200 μl of phosphate buffered saline (pH=7). Genomic DNA was extracted using the QIAGEN DNEASY® Blood and Tissue Kit (QIAGEN, Valencia, CA) or the Promega WIZARD© Genomic DNA Purification Kit (Promega, Madison, Wisconsin) following the manufacturers' recommendations. Illumina based sequencing (Illumina, San Diego, California) was conducted at Novogene (Sacramento, CA). Comprehensive genome analysis (including de novo assembly and annotation) of the isolates was conducted in PATRIC (Wattam et al., 2017, Nucleic Acids Res; 45:D535-D542). The whole genome of the isolates was compared to established vaccine and lab strains using the Similar Genome Finder and Phylogenetic Tree Builder tools in PATRIC.

After preliminary screening of the ten candidates in turkeys, K4110A and K6067 were further assessed regarding safety and efficacy in chickens (Trial 3), the most protective and effective dose and route of administration of each (Trials 4 and 5) and potential vertical transmission of each (Trial 6). R-strain, a well described pathogenic strain of MG (Rodriguez and Kleven, 1980, Avian Dis; 24:800-7) was used to challenge vaccinated or naïve birds as a positive control group. Aerosol and eye drop routes were the two routes of administration used to inoculate the birds; with the former, 1 mL of actively growing culture of the strain was aerosolized using a commercial paint sprayer (PREVAL® Sprayer Division, Precision Valve Corporation, Yonkers, NY) and with the latter, 100 μl of the actively growing culture was administered into the eye.

### Serology

Sera were analyzed to confirm the stimulation of the humoral immune system, the presence of anti-MG antibodies was evaluated by serum plate agglutination (SPA) using commercial antigen (Charles River Laboratories, North Franklin, CT), the hemagglutination (HI) test using antigen prepared from the A5969 strain and chicken red blood cells (RBCs), and enzyme-linked immunosorbent assay (ELISA) using a commercial kit (IDEXX, Westbrook, ME; Biochek, Scarborough, ME). Procedures described previously (Kleven SH, “Mycoplasmosis,” In: Dufour-Zavala L, Swayne D E, Glisson J R, Pearson J E, Reed W M, Jackwood M W. Woolcock P R, editors. A Laboratory Manual for the Isolation, Identification and Characterization of Avian Pathogens, Fifth Edition. American Association of Avian Pathologists. p. 59-64; 2008) were used to perform SPA and HI tests, and scores ≥1 was considered positive for SPA while with HI, 1:20 titer was considered suspect and ≥1:40 titers were considered positive. For ELISAs sample/positive (S/P) ratios ≥0.5 were considered positive.

### Isolation and Identification of Mycoplasma

Cotton swabs were taken from tracheas, choanal clefts and air sacs and inoculated in Frey's modified broth and agar (Ferguson-Noel and Kleven, “Mycoplasma species,” In: Williams S M, Dufour-Zavala L, Jackwood M W, Lee M D, Lupiani B, Reed W M, Spackman E. Woolcock P R, editors. A Laboratory Manual for the Isolation, Identification and Characterization of Avian Pathogens. American Association of Avian Pathologists. p. 63-70; 2016).

For Trials 1 and 2, the samples were also inoculated in modified PPLO broth and agar (Ferguson-Noel and Kleven, “Mycoplasma species,” In: Williams S M, Dufour-Zavala L, Jackwood M W, Lee M D, Lupiani B, Reed W M, Spackman E. Woolcock P R, editors. A Laboratory Manual for the Isolation, Identification and Characterization of Avian Pathogens. American Association of Avian Pathologists. p. 63-70; 2016) pre-inoculation when screening birds for the presence of Mycoplasma spp. All cultures were incubated at 37 C and if no growth was noticed in the broth tubes or agar plates after four weeks, the cultures were considered negative for Mycoplasma. Direct immunofluorescence was used to identify Mycoplasma isolates (Talkington and Kleven, 1983, Avian Dis; 27:422-9).

qPCR and Colonization Rate Calculation

Real-time quantitative PCR (qPCR) procedures for specific detection of MG and MS, and MM and MI were carried out using protocols previously described (Raviv and Kleven, 1983, Avian Dis; 53:103-7; and Callison et al., 2006, Avian Dis; 50:537-44). The qPCR was performed to estimate the genome copy numbers of MG present in tracheal samples collected at necropsy. In addition, strain specific qPCRs were performed for R strain, K4110A and K6067 to evaluate the efficacy of these two vaccine candidates in controlling the colonization of the pathogenic challenge strain. Tracheal swabs were taken during the trials, and at necropsy upper tracheal sections of individual birds were collected in 10 ml sterile PBS at necropsy for later DNA extraction. Genomic DNA was extracted from 200 μl of the laryngeal wash and tracheal swabs using the MAG-BIND® Blood and Tissue DNA HDQ 96 kit (Omega Bio-tek, Inc., Norcross, GA) on the MAGMAX™ Express-96 Magnetic Particle Processors (Thermo Fisher Scientific) following the manufacturer's recommendations. Real-time PCR was performed using an Applied Biosystems 7500 Fast Real-Time PCR System (Thermo Fisher Scientific) and a cycle threshold (Ct) value ≤39 was considered positive. To make the assays quantitative, plasmids were constructed containing the genome target as standard DNA controls. The procedures used in constructing the DNA controls and standard curves for quantitation have been described in detail elsewhere (Raviv et al., 2008, Vet Microbiol; 129:179-87).

### Evaluation of Lesions

Gross air sac lesions were scored on a scale of 0 to 4 using scoring systems described by Kleven (Kleven et al., 1972, Avian Dis; 16:915-24; and Kleven et al., 1975, Avian Dis; 19:126-35). Tracheal lesions were evaluated microscopically by measuring the width of the tracheal mucosa. A section of the upper third of the trachea (approximately 1 inch distal from the larynx) was fixed in 10% neutral formalin. The tracheal mucosa thickness was measured at four equidistant points on histological slides of cross sections of tracheas (Whithear, 1996, Rev. Sci. Tech. Off Int. Epiz. 15:1527-1553). Any other necropsy findings were also recorded.

### Animal Care and Use

All animal procedures in these experiments were approved by the Institutional Animal Care and Use Committee of the University of Georgia, Athens, GA. All birds in these studies were provided with feed and water ad libitum and euthanized with carbon dioxide.

### Statistical Analysis

One-way ANOVA was used to analyze air sac lesions and M. gallisepticum isolations from chickens. Mean tracheal mucosal thickness, MG DNA genome copy number log 10, SPA scores, S/P ratios, HI titers, fertility rate and hen-week egg production rates were analyzed using Tukey-Kramer highly significant difference test (Minitab 17 Statistical Software. [Computer software]. State College, PA: Minitab, Inc. (www.minitab.com). 2010). A P value ≤0.05 was considered significant.

### Experimental Designs

### Trial 1 and 2 (Turkey Safety Land 2)

Trials 1 and 2 had the same experimental design; in each trial 5 candidates were included for a total of 10 vaccine candidate evaluated over the two trials. For each of these trials, 42 turkey poults were acquired at 1 day of age from a source known to be free of MG and MS and housed in 2 isolators. At 2 weeks of age (WOA) the turkeys were leg banded with random numbers (www.randomlists.com) and randomly assigned to 7 treatment groups (7 isolators) of 6 poults each. Ten turkeys, distributed among the treatment groups, were screened for the presence of Mycoplasma by culture, quantitative polymerase chain reaction (qPCR) of tracheal samples and of Mycoplasma antibodies in blood serum at this time. At 3 WOA the turkeys were inoculated via aerosol with their respective treatments or left uninoculated (negative control group) (Tables 2 and 3). At 5 WOA, all birds were necropsied and evaluated by gross air sac lesion scoring, serology, tracheal histopathology, and MG-specific qPCR of tracheal washes.

### Trial 3 (Chicken Safety and Efficacy)

One hundred-twenty-five day-old SPF chicks were acquired from a source known to be free MG and MS and housed in one pen (1.5×3 m²) with pine shavings litter. At 3 WOA, the chicks were randomly wing banded and assigned to 4 treatment groups. Fifteen chickens (distributed among the treatment groups) were screened for the presence of Mycoplasma spp. by culture, MG and M. synoviae qPCR of tracheal samples, and serology. At 4 WOA, the chickens were inoculated via aerosol with their respective treatments (B, C, D, K6067, K4110A or R-strain) or left unvaccinated as the negative control group as detailed below in Table 4. Five chickens in groups B and C were not inoculated and were commingled with the vaccinated birds at 5 WOA. At 19 days post commingling (8 WOA) these 10 contact birds were necropsied and evaluated. At 4 weeks post vaccination 10 chickens each from groups B, C and D were directly challenged with R strain via aerosol. The remaining vaccinated birds were commingled with these birds and 10 naive birds were placed in direct contact with the unvaccinated aerosol challenged group (contact challenge). At 10 WOA all birds were necropsied and evaluated by gross air sac lesion scoring, serology, tracheal histopathology, and MG qPCR and strain-specific qPCR of tracheal washes.

### Trial 4 (Dose Response 1)

Eighty-four day-old SPF chicks were acquired from a source known to be free of MG and MS. At 2 WOA the chicks were wing banded and randomly assigned to 12 treatment groups in twenty-four isolators (Table 5). Ten chicks, distributed among the treatment groups, were screened for the presence of Mycoplasma spp. by culture, MG and M. synoviae qPCR of tracheal samples, and serology. At 3 WOA the chickens in groups C through G were vaccinated via eye drop with K6067 and chickens in groups H through L were vaccinated via aerosol with K6067. As indicated in Table 5, the groups received different doses of the vaccine candidate. Groups A and B were left unvaccinated as negative control and challenge only groups, respectively. At 1 week post vaccination, choanal cleft and tracheal swabs were taken from all groups for culture and MG qPCR. At 2 weeks post vaccination, all eighty-four chickens were bled and swabbed for MG serology, culture, and MG qPCR. Four weeks post vaccination and at 7WOA, chickens of groups B to L were challenged with R-strain via aerosol. At 9 WOA, all birds were necropsied and evaluated by gross air sac lesion scoring, serology, choanal cleft and air sac culture, tracheal histopathology, and MG and strain-specific qPCR of tracheal washes. The minimum infective dose 50 (MID50) and minimum protective dose 50 (MPD50) were calculated (Cottey et al., 2001, Current Protocols in Immunology; 42:19-11) based on sampling and necropsy results.

### Trial 5 (Dose Response 2)

Seventy-nine day-old SPF chicks were acquired from a source known to be free of MG and MS. At 3 WOA the chicks were wing banded and randomly assigned to 7 treatment groups in nineteen isolators (Table 6). Ten chicks, distributed among the treatment groups, were screened for the presence of Mycoplasma spp. by culture, MG, and M. synoviae qPCR of tracheal samples and serology. At 3 WOA the chickens of groups C through G were vaccinated via eye drop with different doses of the vaccine candidate K4110A. Groups A and B were left unvaccinated as negative control and challenge only groups, respectively. At 1 week post vaccination, choanal cleft and tracheal swabs were taken from all groups for culture and MG q-PCR. At 2 weeks post vaccination, all seventy-nine chickens were bled and swabbed for MG serology, culture, and MG qPCR. At 4 weeks post vaccination and at 7 WOA, chickens of groups B through G were challenged with R-strain via aerosol. At 9 WOA (2 weeks post challenge), all birds were necropsied and evaluated by gross air sac lesion scoring, serology, choanal cleft and air sac culture, tracheal histopathology, and MG and strain-specific qPCR of tracheal washes. The experimental design and specific dates of the procedures are detailed in Table 6.

The minimum infective dose 50 (MID50) and minimum protective dose 50 (MPD50) were calculated (Cottey et al., 2001, Current Protocols in Immunology; 42:19-11) based on sampling and necropsy results.

### Trial 6 (Vertical Transmission)

One hundred and forty-four 4 week old SPF chickens, (131 females and 18 males), were acquired and housed in four pens (1.5×3 m²) with pine shavings litter. At 19 WOA, the chickens were moved to colony houses and randomly assigned to 5 groups (Table 7). Sixteen chickens, distributed among the treatment groups, were screened for the presence of Mycoplasma spp. by culture, MG and M. synoviae qPCR of tracheal samples, and serology at 23 WOA. At 25 WOA, the chickens were inoculated via eye drop with their respective treatments (K6067, K4110A, F strain or R strain) or left uninoculated as the negative control group. Three weeks post-inoculation at 28 WOA, MG qPCR was performed on choanal cleft swabs from five birds of the negative control group, group A, and 10 birds of each groups B through E. Eggs were collected daily from one week prior to inoculation (24 WOA) to 31 WOA and kept at 4 C until beginning incubation. Eggs laid on weekdays except Mondays were set on Saturdays and egg laid on Saturdays, Sundays and Mondays were set on Mondays. Eggs of each set were candled on days 7, 9, 11, 13 and 15 days of embryonation (DOE). Infertile eggs or early dead embryos (less than 7 DOE) were discarded the yolk sac of any dead embryonated eggs at 9, 11, 13 and 15 DOE were sampled and inoculated into modified Frey's broth for culture. All remaining embryonated eggs were sampled for culture at 18 DOE.

In addition, HI and ELISA (IDEXX, Westbrook, Marine) assays were performed on yolk sac samples of the last set of eggs (National Poultry Improvement Plan, “Procedures for preparing egg yolk samples for diagnostic tests,” National Poultry Improvement Plan Program Standards, 23-24; 2017). The standard procedure recommended by NPIP for egg yolks was used to prepare the samples and perform the tests (National Poultry Improvement Plan, “Procedures for preparing egg yolk samples for diagnostic tests,” National Poultry Improvement Plan Program Standards, 23-24; 2017). The HI test procedure were as described by Ferguson-Noel et al. (Ferguson-Noel and Kleven, “Mycoplasma species,” In: Williams S M, Dufour-Zavala L, Jackwood M W, Lee M D, Lupiani B, Reed W M, Spackman E. Woolcock P R, editors. A Laboratory Manual for the Isolation, Identification and Characterization of Avian Pathogens. American Association of Avian Pathologists. p. 63-70; 2016). Egg production parameters, including the fertility and hen-week production rates were also recorded for all groups during the laying period. The fertility rates of all groups for 16 sets of eggs were calculated by dividing the number of fertile eggs after 7 DOE divided by the total number of incubated eggs times 100 (Lin and Kleven, 1982, Avian Dis; 26:487-95). The number of eggs laid were recorded from 21 to 31 WOA and hen-week egg production rates were also calculated by dividing total number of eggs laid on the week by the total number of hens times 100.

At 31 WOA, all birds were necropsied and evaluated by gross air sac lesion scoring, serology, choanal cleft and air sac culture, tracheal histopathology, and MG qPCR of tracheal washes and oviduct swabs.

### Results

### Trial 1 (Turkey Safety 1)

The birds tested pre-inoculation at 2 WOA were all negative for MG, MM and MS antibodies and were also negative for MG, MS, MM and MI by qPCR. All cultures were also negative for Mycoplasma spp. at that time. MG antibodies were detected in all of the inoculated groups; the serology results of Trial 1 are summarized in Table 8. The weakest antibody response was in Group E, which was inoculated with isolate K6837. The only group in which no air sac lesions were observed was the negative control group, however, the groups inoculated with K6067 (Group B) and K6837 (Group E) had significantly lower mean air sac lesion scores than the positive controls (Group G) (P<0.05). The highest mean tracheal mucosal thickness measurements were in Groups D and F. The only inoculated groups with mean measurements lower than the positive control (R-strain; Group G) were inoculated with K6067 (Group B) and K6837 (Group E). The qPCR results indicated that all of the inoculated groups were infected with MG and that MG was replicating at high levels in the tracheas, with the exception of Group E (K6837 inoculated). Mycoplasma was recovered from all of the cultures (trachea and air sacs) from the inoculated groups with the exception of Group E (inoculated with K6837) and 2 air sac cultures from Group B (inoculated with K6067) (Table 9). There was no mortality during the trial, but the clinical signs observed are summarized in Table 10. No respiratory clinical signs were observed in the birds in Group B (inoculated with K6067) and Group E (inoculated with K6837).

### Trial 2 (Turkey Safety 2)

The 10 birds tested pre-inoculation at 3 WOA were negative for MG, MM and MS antibodies and were also negative for MG, MS, MI, and MM by qPCR. All cultures were also negative for Mycoplasma spp. MG antibodies were detected in all of the inoculated groups and the weakest antibody response was in Group C, which was inoculated with K4110A (Table 11). This was consistent with the mean air sac lesion scores. The only group in which no air sac lesions were observed was the negative control group. However, the groups inoculated with K4110A (Group C) and K4179 (Group B) had lower mean air sac lesion scores than the positive controls (Group G). The difference between the mean air sac lesion scores of group B, (3.5) and the positive control (3.7) was not significant (P<0.05). The highest mean tracheal mucosal thickness measurements were in Groups B and F. No mean measurement any inoculated group was lower than the positive control (Group G). The qPCR results indicated that all inoculated groups were infected with MG and that MG was replicating at high levels in the tracheas. These results also indicated that although K4110A inoculation did not result in significant lesions (P<0.05), the birds were infected and replicated the MG. Mycoplasma was recovered from all cultures of the inoculated groups, both from trachea and air sacs. These results are summarized in Table 12. There was no mortality during the trial, but the clinical signs observed are summarized in Table 13. No respiratory clinical signs were observed in the birds in Group C (inoculated with K4110A) and Group F (inoculated with K6836A).

### Trial 3 (Chicken Safety and Efficacy)

The 15 birds tested pre-inoculation at 2 WOA were negative for MG and MS antibodies and were also negative for MG and MS by qPCR. All cultures were also negative for Mycoplasma spp. pre-inoculation. Three necropsies were performed in this trial with the aim of evaluating the safety of the vaccine candidates in chickens (Necropsy 1), evaluating the horizontal transmission possibility of the vaccine candidates (Necropsy 2), and evaluating the efficacy of the vaccine candidates post challenge (Necropsy 3).

Necropsy 1 (Safety). MG antibodies were detected in all inoculated groups although the antibody response in the groups inoculated with the vaccine candidates was weaker compared to the positive control (R strain). The mean SPA scores of groups B (K6067) and C (K4110A) were significantly lower than the scores of group D (R strain) (P<0.05). A summary of these serology results is presented in Table 14. All groups except the positive control group had no air sac lesions at 14 days post inoculation and these differences were statistically significant (P≤0.05). There were no significant differences in mean tracheal mucosal thickness among the groups (P≤0.0.) although the positive control (R strain) was numerically higher MG was isolated from the tracheas of all inoculated birds at 14 days post inoculation, but no MG was isolated from the air sacs of the vaccine candidate groups as compared to MG isolation from 100% of the R-strain group. The qPCR results showed that although all of the isolates colonized birds in their groups, there was significantly lower genome copies (MCNlog10) in the K4110A compared to the groups inoculated with K6067 and R-strain (P≤0.05). A summary of these results is presented in Table 15.

Necropsy 2 (Horizontal Transmission to Contacts). There was a low level of infection in birds placed in direct contact with vaccinated birds at 19 days post commingling (2 of 5 for K6067 and 1 of 5 for K4110A detected by isolation from the trachea). There were no significant differences in serological response (Table 16), air sac lesions or tracheal mucosa thickness between the groups (Table 17).

Necropsy 3 (Efficacy—Aerosol and Contact Challenge). Groups inoculated with the vaccine candidates showed a strong serological response at 14 days post challenge with R-strain (presented in Table 18). As can be seen in Table 19, the vaccinated groups also had significantly lower mean air sac lesion scores after challenge (P≤0.05). There were no significant differences with respect to tracheal measurements (Table 21), even between the negative and positive controls (P≤0.05); however, the positive control resulted in the highest mean measurement. The qPCR resulted are summarized in Table 20. The qPCR results show that the K6067 group had higher mean genome copy numbers log 10 in their tracheas compared to the K4110A groups. It is also evident that most of the MG detected in the tracheas of the vaccinated groups was the vaccine strain not the challenge strain for both vaccine candidates. It appeared that both vaccines were able to protect against colonization with the challenge strain with significantly lower MCNlog10 for R-strain specific qPCR in the vaccinated groups (P<0.05). For the contact challenged groups, there was some transmission of R-strain to the non-vaccinated contact birds, and we can infer that at least 70% (7/10) of these contact birds were infected with R-strain from the isolation results but none of the vaccinated birds exposed to R-strain by contact with the challenged birds were infected with the challenge strain (from the R-strain qPCR results).

### Trial 4 (Dose Response 1)

All 10 birds tested pre-inoculation at 2 WOA were negative for MG and MS antibodies and were also negative for MG and MS by qPCR. All cultures were also negative for Mycoplasma spp. at this time. A summary of findings of the sampling at 4 WOA (1 week post vaccination) is presented in Table 22. All cultures of groups E, F and G (doses 10³, 10⁴, and 10⁵ CCU/mL via eyedrop respectively) were positive for Mycoplasma spp. In the other groups, only one sample in each of group D (10² CCU/mL via eyedrop), J and K (10⁵ and 10⁶ CCU/mL via aerosol) were positive. The qPCR results also indicated some MG colonization only in groups E, F and G with K6067.

The results sampling at 6 WOA (3-weeks post vaccination) is presented in Tables 23 and 24. All cultures of groups E, F and G were again positive for Mycoplasma one sample each from groups J and K and two samples of group D were positive and the rest were all negative. MG colonization was also evaluated by performing qPCR on tracheal swabs and the groups D, E, F and G showed some level of colonization, with the group F (10⁴ CCU/mL via eyedrop) having a colonization rate was 100%. In case of serology, the serum plate agglutination (SPA) test indicated some antibody response in groups D through G, J and K but with hemagglutinin inhibition (HI) and enzyme-linked immunosorbent assay (ELISA) testing, there were few positives in group F and G. At 6 weeks post vaccination (2 weeks post challenge with R-strain) MG antibodies were detected in all inoculated groups (B to G) (Tables 25 and 26). There were lower mean SPA scores in groups E through G (which all showed K6067 colonization in trachea in earlier samplings) compared to group B (the challenge-only group). All groups except the negative control group, (group A), had some level of air sac lesions after virulent challenge but the mean air sac lesion scores of groups D, E, F, G, J, K and L were lower than challenge only group (Group B). Of those, the mean scores of groups E, F and G were significantly lower compared to the other vaccinated and challenged groups (P≤0.05). The mean tracheal mucosal thickness measurements of groups D, E, F, G, J and K were lower compared to the challenge only group and the difference was significant with groups E, F and G (P<0.05) (Table 27).

The qPCR results, summarized in Table 28, indicated that all inoculated groups were infected, and MG was replicating in the tracheas. These mean colonization rates of groups E, F and G, (which were vaccinated with the highest doses of K6067 via eye drop) were significantly lower than other groups including the challenged-only group (Group B) (P<0.05). The mean colonization rates of groups D, J and K were also lower than group B. Strain-specific qPCR results correlated with MG qPCR and for the groups (Table 28). Mycoplasma was recovered from all choanal cleft cultures from the inoculated groups after R-strain challenge, except two birds of Group J. For air sac cultures, Mycoplasma was recovered from the air sacs of all groups vaccinated via aerosol, (groups H to L). With groups vaccinated via eye drop, Mycoplasma was isolated from all birds of groups C, D and G, three birds of group E and five birds of group F (Table 27). The MID50 for K6067 was calculated based on the qPCR and culture results of 1- and 3-weeks post vaccination sampling for all doses and both routes of administration. While no MID50 could be calculated for qPCR results, the MID 50 for the culture results of groups vaccinated via eye drop route was 10^{3.8} ccu/mL one week post vaccination and 10^{3.53} CCU/mL at three weeks post vaccination. No MID50 could be calculated for from the culture results of the groups vaccinated via aerosol route (Table 29).

In addition, MPD50 for K6067 was also calculated based on air sac scores and tracheal thickness measurements recorded at the necropsy. While no MPD50 could be calculated for with either the air sac scores and tracheal thickness measurements of groups vaccinated via aerosol, the MPD50 for groups vaccinated via eye drop was 10⁴ CCU/mL with air sac scores and 10^{3.3} CCU/mL with tracheal thickness measurements (Table 30).

### Trial 5 (Dose Response 2)

All 10 birds tested pre-inoculation at 2 WOA were negative for MG and MS antibodies and were also negative for MG and MS by qPCR. All cultures were also negative for Mycoplasma spp. at that time. A summary of findings from the sampling at 4 WOA (1 week post vaccination) is presented in Table 31. Ten out of 13 cultures of group C and all cultures from Groups D to G were positive for Mycoplasma. The qPCR results also indicated some levels of MG colonization in all groups inoculated with K4110A, with groups F and G (the groups that received the highest vaccine doses) having significantly higher mean genome copy numbers (MCNlog10) than the negative controls (P<0.05).

The results from the sampling at 6 WOA (3 weeks post vaccination) are summarized in Tables 32 and 33. All cultures of inoculated groups (except 2 birds in group C) were positive for Mycoplasma. Some level of colonization was detected in all of the inoculated groups with groups E, F and G having significantly higher mean genome copy numbers (MCNlog10) than the negative controls (P<0.05). With respect to serology, more positive samples were found with the SPA test compared to the HI and ELISA tests. The SPA scores of group C (inoculated with the lowest dose of the vaccine) were significantly lower than other inoculated groups (P<0.05). Group C also had no positive results on the ELISA and HI tests.

At 9 WOA (6 weeks post vaccination and 2 weeks post challenge with R-strain) seroconversion was observed in all inoculated groups. The mean SPA scores of groups C through G were lower compared to Group B (the challenge-only group). However, s/p ratios and HI titers from these groups were all higher than Group B (Table 34). All of the groups had some air sac lesions except groups A (negative controls) and F. However, the mean air sac lesion scores of all vaccinated groups were significantly lower than Group B (challenge only controls) (P<0.05) (Table 35). The mean tracheal mucosal thickness measurements of all vaccinated groups were significantly lower compared to the challenged only group (Group B) (P<0.05). Mycoplasma was isolated from all choanal cleft cultures of all vaccinated of challenged groups and from the air sacs of all birds of all vaccinated of challenged except two birds in group D and one bird in group F.

The MG qPCR and strain specific qPCR results are summarized in Table 36. The R strain mean genome copy number (MCNlog10) detected in the trachea was significantly higher in the challenge-only group compared to the vaccinated groups (P<0.05). K4110A specific qPCR showed that the vaccine candidate colonized the trachea of all K4110A-vaccinated groups although the MCNlog10 of the vaccine strain in Group C (lowest vaccination dose) was significantly lower than the other vaccinated groups. Except for Group G, K4110A was not detected in the air sac cultures from the vaccinated groups (Table 36). Based on qPCR and culture results of 1 and 3 weeks post vaccination for all doses the MID50 for K4110A was calculated (Table 37). While no MID50 was found with culture results, the MID 50 for the qPCR results of groups vaccinated via eye drop route was 10⁵ CCU/mL one week post vaccination and 10^{5.2} CCU/mL at three weeks post vaccination.

The MPD50 for K4110A was also calculated based on mean air sac scores necropsy and was 10^{3.7} CCU/mL. (Table 38). No MPD50 could be calculated the tracheal thickness measurements of vaccinated groups.

### Trial 6 (Vertical Transmission)

All 16 birds tested pre-inoculation at 23 WOA were negative for MG and MS antibodies and were also negative for MG and MS by qPCR. All cultures were also negative for Mycoplasma spp. at that time.

A total of 4436 eggs were tested and none was positive for Mycoplasma. However, HI and ELISA results on egg yolk samples indicated transfer of antibody to the embryos (Table 39). While there were some positive MG ELISA results for groups C (K4110A) D (F-strain) and E (R-strain), only groups C and E were positive in MG HI. The Group B yolk sac samples were not positive by either HI or ELISA. The fertility rates and hen-week production means of all groups for 16 sets of eggs are presented in Table 40; there were no significant differences among the groups (P<0.05).

A summary of the serology results at necropsy is presented in Table 41. MG antibodies were detected in all inoculated groups. No significant differences were noticed with SPA scores of the inoculated groups, although Group B (K6067) HI titer and ELISA S/P ratio were significantly lower than other inoculated groups (P<0.05).

The mean air sac lesions in the groups inoculated with the two vaccine candidates and F strain was significantly lower than group E (R-strain) (Table 42) P<0.05). The highest mean tracheal mucosal thickness measurements were in Group E inoculated with R strain. With respect to tracheal lesions both vaccine candidates (groups B and C) and the group inoculated with F strain (group D) had significantly lower mean measurements than the positive controls (Group E-R-strain) P<0.05). MG qPCR results of tracheal wash samples indicated that all inoculated groups were infected with MG and that MG was replicating in the tracheas. These results showed that MG colonization rates in the trachea of groups inoculated with both vaccines candidates were higher than groups the groups inoculated with F-strain and R-strain. None of the oviduct swabs were positive by MG q-PCR. Mycoplasma was recovered from all choanal cleft cultures of all the inoculated groups except one bird from group C and another from group D (Table 42). Both negative samples were from birds that were positive in q-PCR and air sac culture. The number of positive air sac cultures in all the vaccinated groups were significantly lower than group E (inoculated with R strain).

### Discussion

Ten MG isolates that met our initial criteria for vaccine candidates were first administered to turkeys in Trial 1 and 2. The rationale for selecting isolates recovered from turkeys as well as performing the first safety trials in turkeys was based on the fact that, in general, turkeys are more susceptible to MG and are more severely affected by the disease compared to chickens. The overall goal of this research was to develop a live attenuated MG vaccine candidate that is both effective and safe in both turkeys and chickens. It was supposed that vaccine candidates that have some degree of natural attenuation would be more promising candidates than virulent strains. Analysis of results (clinical signs, air sac and tracheal lesions) from the two turkey trials led to the selection of K6067 and K4110A for further study as these isolates showed evidence of being naturally attenuated (to some degree). There was another isolate in Trial 1 (K6837), that resulted in the weakest antibody response and low levels of lesions compared to the other inoculated groups. However, the inoculation titer of this isolate (10⁶ CCU/ml) and was comparatively much lower than the inoculation titer of the other isolates (10⁸ CCU/ml) and it appeared that this isolate did not colonize the turkeys as well as the other MG isolates based on the qPCR results. Although K6837 met the minimum requirement for robust in vitro growth, it was the slowest growing of the isolates selected for this trial and this isolate will require further study to determine if the results from Trial 1 were due to the low titer (and low colonization rate) or low virulence of the isolate.

The results of Trial 1 indicate that although K6067 inoculation did not result in significant lesions the turkeys were infected and replicated the MG (a requirement to generate a protective immune response). It has been shown that 6/85 poorly colonizes the trachea and this may explain the compared to F-strain and ts-11 (16, 29). The air sac culture results also indicated that K6067 may not be as invasive as the other MG isolates in this trial. Similarly, in Trial (Turkey Safety 2) K4110A was capable of infecting turkeys but did not result in severe clinical signs or lesions. All of the remaining trials were conducted in chickens although further research in turkeys is necessary to investigate efficacy and other parameters in this poultry species.

In Trial 3, the two selected candidates were assessed for safety (including the potential for horizontal transmission) and efficacy in chickens. In the first part of this trial, it was determined that both vaccine candidates met the requirements for safety in chickens, as although the isolates colonized the respiratory system and generated a systemic antibody response there were no clinical signs, and no air sac lesions after administration of K6067 or K4110A by aerosol. In this trial K6067 was present at higher levels in tracheal samples compared to K4110A, but this may have been due to the slightly lower dose of K4110A administered (10⁷ CCU/ml vs 10⁸ CCU/ml). Another aspect of safety—the risk of horizontal transmission was evaluated by commingling the vaccinated birds with naïve contacts. There was some evidence of infection in the contacts but as the birds were commingled for 14 days only, the transmissibility of the vaccine candidates should be investigated more thoroughly before conclusions are made. With respect to efficacy of the vaccine candidates, the results from this trial following virulent challenge with MG R-strain indicated that both candidates induced protective immunity in chickens. The vaccinated birds had significantly lower mean air sac scores compared to the unvaccinated controls for the birds that were challenged by aerosol.

Although there were no significant differences in tracheal mucosa measurements among the groups in this trial this is likely due to combined data from aerosol challenged and contact challenged birds. The contact challenged birds did not have severe lesions, even in the control group that was not vaccinated. This challenge method is not generally expected to result in strong challenge with high lesion scores however, the qPCR results indicate that the vaccine candidates were able to prevent (with contact infection) or reduce (with direct aerosol challenge) infection and colonization with the challenged strain. Further investigation of the efficacy of these candidates (including direct comparison to commercial vaccines) should be conducted to confirm these preliminary results. However, the results of Trials 4 and 5, indicate that the vaccine candidates have minimum infectious doses and protective doses comparable to the commercial vaccines when administered by the eye drop route. A dose in the range of 10³ CCU/ml via eye drop was sufficient to infect 100% (for K6067) and 77% (for K4110A) of birds at 1 week post vaccination. This dose was also sufficient to provide some protection against air sac and tracheal lesions following virulent challenge. The minimum effective dose for F strain was calculated at 10⁵ CCU/mL (Lin and Kleven, 1984, Avian Dis; 28:273-7). Aerosol administration of K6067 was not very successful; equipment and environmental complications with aerosol administration of the vaccine to the small numbers of birds in each group in the trial are likely to have affected this part of the study. More investigations (with greater numbers of birds) may show that aerosol can be an effective administration route, but from the results of this trial it appears that the eye drop route is preferred for K6067 vaccination. Similar these results eye drop was found to be the most effective route of administration for F strain (Leigh et al., 2018, Poultry Science; 97(9):3072-3075; and Evans et al., 2015, Poult Sci; 94:1849-52). More consistent results and increased immune responses measured by serology) for the eye drop vaccinated group. The better results seen with the eye drop route may be due to several factors, including individual administration of the vaccine and more consistent volumes and doses for each bird in the flock. It should be noted that in Dose Response 2 (K4110A) strain specific PCRS were not only conducted on tracheal samples but also on air sac cultures and the genome copy numbers were very high for R-strain; this is likely due to the adaptation of the R-strain to in vitro culture.

In both turkeys and chickens, the two vaccine candidates invoked a detectable and consistent serological response; although serum antibody levels do not appear to correlate with protection for MG live attenuated vaccines it is useful to be able to monitor vaccination using widely available and inexpensive serological tests.

Vertical transmission can become a major safety concern for Mycoplasma vaccines and both F-strain and ts-11 vaccine have been shown to have some risk of transmission to progeny through the egg (Armour et al., 2015, Avian Pathol; 44:296-304; and El Gazzar et al., 2011, Avian Dis; 55:569-74). No vertical transmission was found with either candidate in this research. However, vertical transmission was also not detected in groups inoculated with F strain or R strain. These results may be due to the relatively small numbers of birds in the trial and also the route of administration (eye drop). While this route more closely mimics the likely field application of the vaccine, higher transmission rates may be seen with a more severe challenge (aerosol). The results indicate that the vaccine candidates are unlikely to have a high rate of vertical transmission but increasing the numbers of birds and numbers of eggs should be considered to confirm these results. Detection of the transfer of maternal antibodies in the egg yolk samples indicated some systemic immune response to the MG. The role of passive maternal antibody in MG infections has been shown to be not beneficial as it provides little protection against MG challenge (Lin and Kleven, 1984, Avian Dis; 28:79-87) and embryo mortality caused by virulent MG is blocked by this antibody (Levisohn et al., 1985, Avian Dis; 29:188-97) and as the result, there may be an increase the hatchability of infected eggs which is not ideal.

In conclusion, both candidates were safe in turkeys and chickens and effective in reducing the colonization with the virulent challenge strain as well as and reducing the lesions and effects of the virulent MG strain in chickens. The eye drop route was confirmed as an effective route of administration and the infectious and protective doses correspond to other MG vaccines. The vertical transmission study indicated that neither K6067 nor K4110A has a high risk of transmission. These results are promising and further studies evaluating parameters such as duration of immunity, transmissibility, and efficacy in turkeys will provide further information on these vaccines.

TABLE 1
The history of vaccine candidates selected
for preliminary screening (Trials 1 and 2).

| # | K# | Isolation history | Trial |
|---|---|---|---|
| 1 | K6837 | Turkeys, North Carolina, 2017 | Trial 1 |
| 2 | K6813 | Turkeys, North Carolina, 2017 | Trial 1 |
| 3 | K6694 | 51 WOA broiler breeders, Arkansas, 2015 | Trial 1 |
| 4 | K6524 | Turkey breeders, Minnesota, 2012 | Trial 1 |
| 5 | K6067 | 19 WOA turkeys, Arkansas, 2007 | Trial 1 |
| 6 | K6836A | Turkeys, North Carolina 2017 | Trial 2 |
| 7 | K5792A | Layer breeders, Pennsylvania, 2005 | Trial 2 |
| 8 | K5058E | Turkeys, Virginia, 2001 | Trial 2 |
| 9 | K4179 | 27 WOA turkey breeders, Virginia, 1996 | Trial 2 |
| 10 | K4110A | 73 WOA turkeys, North Carolina, 1996 | Trial 2 |

WOA = weeks of age

TABLE 2
Trial 1 (Turkey Safety 1). Experimental Design.

| | | | | Titer of |
|---|---|---|---|---|
| | No. of Birds | Vaccination | Passage | Culture |
| Group | per Isolator | (spray) | level | (CCU/ml) |
| A | 6 | No | N/A | N/A |
| B | 6 | Yes/K6067 | 13P | 8.0 × 10⁸ |
| C | 6 | Yes/K6524 | 11P | 4.2 × 10⁸ |
| D | 6 | Yes/K6694 | 10P | 3.9 × 10⁸ |
| E | 6 | Yes/K6837 | 11P | 3.1 × 10⁶ |
| F | 6 | Yes/K6813 | 14P | 1.8 × 10⁸ |
| G | 6 | R-strain | 12P | 3.5 × 10⁸ |

TABLE 3
Trial 2 (Turkey Safety 2). Experimental Design.

| | | | | Titer of |
|---|---|---|---|---|
| | No. of Birds | Vaccination | Passage | Culture |
| Group | per Isolator | (spray) | level | (CCU/ml) |
| A | 6 | No | N/A | N/A |
| B | 6 | Yes/K4179 | 7P | 8.0 × 10⁸ |
| C | 6 | Yes/K4110A | 10P | 4.2 × 10⁸ |
| D | 6 | Yes/K5058E | 11P | 3.9 × 10⁸ |
| E | 6 | Yes/K5792A | 12P | 3.1 × 10⁶ |
| F | 6 | Yes/K6836A | 13P | 1.8 × 10⁸ |
| G | 6 | R-strain | 12P | 3.5 × 10⁸ |

TABLE 4
Trial 3 (Chicken Safety and Efficacy). Experimental Design.

| | | | | Titer of |
|---|---|---|---|---|
| | No. of Birds | Vaccination | | Culture |
| Group | per group | (spray) | Passage level | (CCU/ml) |
| A | 18 | No | N/A | N/A |
| B | 38 | Yes/K6067 | 13P | 4.2 × 10⁸ |
| C | 38 | Yes/K4110A | 11P | 2.3 × 10⁷ |
| D | 31 | Yes/R strain | 13P | 2.8 × 10⁸ |

Note:
The titer of R strain 13P used for challenging groups B, C and D after the inoculation was 2.45 × 10⁹ CCU/ml.

TABLE 5
Trial 4 (Dose Response 1). Experimental Design.

| | No. of Birds | | | Dose & route |
|---|---|---|---|---|
| Group | per group | Vaccination | Challenge | of vaccination |
| A | 7 | No | No | N/A |
| B | 7 | No | Yes/R 12P | N/A |
| C | 7 | Yes/K6067 | Yes/R 12P | 10¹ CCU/mL |
| | | 18P | | eye drop |
| D | 7 | Yes/K6067 | Yes/R 12P | 10² CCU/mL |
| | | 18P | | eye drop |
| E | 7 | Yes/K6067 | Yes/R 12P | 10³ CCU/mL |
| | | 18P | | eye drop |
| F | 7 | Yes/K6067 | Yes/R 12P | 10⁴ CCU/mL |
| | | 18P | | eye drop |
| G | 7 | Yes/K6067 | Yes/R 12P | 10⁵ CCU/mL |
| | | 18P | | eye drop |
| H | 7 | Yes/K6067 | Yes/R 12P | 10³ CCU/mL |
| | | 18P | | aerosol |
| I | 7 | Yes/K6067 | Yes/R 12P | 10⁴ CCU/mL |
| | | 18P | | aerosol |
| J | 7 | Yes/K6067 | Yes/R 12P | 10⁵ CCU/mL |
| | | 18P | | aerosol |
| K | 7 | Yes/K6067 | Yes/R 12P | 10⁶ CCU/mL |
| | | 18P | | aerosol |
| L | 7 | Yes/K6067 | Yes/R 12P | 10⁷ CCU/mL |
| | | 18P | | aerosol |

Note:
The titer of R strain 12P used for challenging groups B to L after the inoculation was 1.1 × 10⁹ CCU/ml.

TABLE 6
Trial 5 (Dose Response 2). Experimental Design.

| | No. of Birds | | | Dose & route |
|---|---|---|---|---|
| Group | per group | Vaccination | Challenge | of vaccination |
| A | 7 | No | No | N/A |
| B | 7 | No | Yes/R 14P | N/A |
| C | 13 | Yes/K4110A | Yes/R 14P | 10³ CCU/mL |
| | | 14P | | eye drop |
| D | 13 | Yes/K4110A | Yes/R 14P | 10⁴ CCU/mL |
| | | 14P | | cyc drop |
| E | 13 | Yes/K4110A | Yes/R 14P | 10⁵ CCU/mL |
| | | 14P | | eye drop |
| F | 13 | Yes/K4110A | Yes/R 14P | 10⁶ CCU/mL |
| | | 14P | | eye drop |
| G | 13 | Yes/K4110A | Yes/R 14P | 10⁷ CCU/mL |
| | | 14P | | eye drop |

Note:
The titer of R strain 12P used for challenging groups B to L after the inoculation was 3.5 × 10⁸ CCU/ml.

TABLE 7
Trial 6 (Vertical Transmission). Experimental Design.

| | | | | Titer of |
|---|---|---|---|---|
| | No. of Birds | Vaccination | Passage | Culture |
| Group | per group | (spray) | level | (CCU/ml) |
| A | 16 | No | N/A | N/A |
| B | 32 | Yes/K6067 | 18P | 4.21 × 10⁸ |
| C | 32 | Yes/K4110A | 14P | 9.3 × 10⁸ |
| D | 32 | Yes/F strain | 11P | 1.77 × 10⁹ |
| E | 32 | Ycs/R strain | 14P | 7.95 × 10⁸ |

TABLE 8
Trial 1 (Turkey Safety 1). MG serological response
of turkeys at 2 weeks post inoculation with K6067,
K6524, K6694, K6837, K6813 or R strain.^{A}

| | Vaccine/ | | | | | | |
|---|---|---|---|---|---|---|---|
| Group | Challenge | SPA | | HI | | ELISA | |
| A | None | 0/6 ^{B} | (0.0)^{C}c | 0/6 | (0.0)^{D}d | 0/6 | (0.0)^{E}a |
| B | K6067 | 6/6 | (1.7)a | 5/6 | (1.2)abc | 3/6 | (0.6)c |
| C | K6524 | 6/6 | (2.2)a | 2/6 | (0.4)cd | 5/6 | (0.8)bc |
| D | K6694 | 6/6 | (2.0)a | 6/6 | (1.5)ab | 6/6 | (1.3)a |
| E | K6837 | 1/6 | (0.2)bc | 3/6 | (0.7)bcd | 0/6 | (0.1)a |
| F | K6813 | 6/6 | (1.2)ab | 5/6 | (2.0)a | 6/6 | (1.3)ab |
| G | R strain | 6/6 | (2.2)a | 6/6 | (1.8)a | 6/6 | (1.2)ab |

^{A}Values within a column at a specific time point with different lowercase letters are significantly different (P ≤ 0.05)
^{B} No. of positive samples/No. of tested samples (SPA: ≥1, HI: ≥20, ELISA: ≥0.5, Air sac score ≥ 1)
^{C}Mean agglutination grade (from 0 to 4)
^{D}Mean titer log10
^{E}Mean sample/positive ratio

TABLE 9
Trial 1 (Turkey Safety 1). Lesions scores, tracheal mucosal
thickness, genome copy numbers log10 and MG isolation from
tracheas and airsacs of turkeys at 2 weeks post inoculation
with K6067, K6524, K6694, K6837, K6813 or R strain.^{A}

| | | Air sac | Tracheal | MG isolation | | |
|---|---|---|---|---|---|---|
| | Vaccine/ | lesion | mucosal | | Air | qPCR − |
| Group | Challenge | score | thickness | Trachea | sacs | MG |
| A | None | 0/6^{B} | 111.7 ± | 0/6a | 0/6a | 0.0^{E}b |
| | | (0.0)^{C}c | 20.7^{D}c | | | |
| B | K6067 | 6/6 | 125.2 ± | 6/6b | 4/6b | 2.5 ± |
| | | (1.5)b | 14.1c | | | 0.2a |
| C | K6524 | 6/6 | 153.6 ± | 6/6b | 6/6b | 3.7 ± |
| | | (3.0)a | 31.1bc | | | 0.9a |
| D | K6694 | 6/6 | 215.5 ± | 6/6b | 6/6b | 3.4 ± |
| | | (3.2)a | 56.9ab | | | 0.7a |
| E | K6837 | 3/6 | 105.1 ± | 5/6b | 0/6a | 1.0 ± |
| | | (0.5)bc | 27.4c | | | 0.7b |
| F | K6813 | 6/6 | 226.5 ± | 6/6b | 6/6b | 3.5 ± |
| | | (3.2)a | 39.6a | | | 0.7a |
| G | R strain | 6/6 | 148.0 ± | 6/6b | 6/6b | 2.9 ± |
| | | (3.7)a | 47.6c | | | 1.2a |

^{A}Values within a column at a specific time point with different lowercase letters are significantly different (P ≤ 0.05)
^{B}No. of positive samples/No. of tested samples (Air sac score ≥ 1)
^{C}Mean score (Macroscopically scored from 0 to 4)
^{D}Mean thickness for the group ± SD (μm)
^{E}Mean (genome) copy number log10 ± SD

TABLE 10
Trial 1 (Turkey Safety 1). Clinical signs in turkeys at 2 weeks post
inoculation with K6067, K6524, K6694, K6837, K6813 or R strain.

| Group | Vaccine/Challenge | Clinical Picture |
|---|---|---|
| A | None | No respiratory clinical signs observed |
| B | K6067 | No respiratory clinical signs observed |
| C | K6524 | Rales and coughing |
| D | K6694 | Gasping, rales and coughing |
| E | K6837 | No respiratory clinical signs observed |
| F | K6813 | Rales and coughing |
| G | R strain | Weak rales |

TABLE 11
Trial 2 (Turkey Safety 2). MG serological responses
of turkeys at two weeks post inoculation with K4179,
K4110A, K5058E, K5792A, K6836A or R strain.^{A}

| | Vaccine/ | | | | | | |
|---|---|---|---|---|---|---|---|
| Group | Challenge | SPA | | HI | | ELISA | |
| A | None | 0/6 ^{B} | (0.0)^{C}c | 0/6 | (0.0)^{D}c | 0/6 | (0.0)^{E}c |
| B | K4179 | 6/6 | (2.3)a | 6/6 | (1.9)a | 6/6 | (1.05)ab |
| C | K4110A | 6/6 | (2.2)ab | 6/6 | (1.8)a | 2/6 | (0.48)bc |
| D | K5058E | 6/6 | (1.3)ab | 6/6 | (1.45)b | 3/6 | (0.40)bc |
| E | K5792A | 6/6 | (1.2)b | 6/6 | (1.9)a | 2/6 | (0.73)bc |
| F | K6836A | 6/6 | (1.7)ab | 6/6 | (1.8)a | 6/6 | (1.59)a |
| G | R strain | 6/6 | (1.8)ab | 6/6 | (1.78)a | 4/6 | (0.91)b |

^{A}Values within a column at a specific time point with different lowercase letters are significantly different (P ≤ 0.05)
^{B} No. of positive samples/No. of tested samples (SPA: ≥1, HI: ≥20, ELISA: ≥0.5, Air sac score ≥ 1)
^{C}Mean agglutination grade (from 0 to 4).
^{D}Mean titer log10
^{E}Mean sample/positive ratio

TABLE 12
Trial 2 (Turkey Safety 2). Lesion scores, tracheal mucosal
thickness, MG isolation from tracheas and air sacs and
qPCR from turkeys at two weeks post inoculation with K4179,
K4110A, K5058E, K5792A, K6836A or R strain.^{A}

| | | Air sac | Tracheal | | | |
|---|---|---|---|---|---|---|
| | Vaccine/ | lesion | mucosal | MG isolation | | qPCR − |
| Group | Challenge | score | thickness | Trachea | Air sacs | MG |
| A | None | 0/6^{B} | 77.12 ± | 0/6a | 0/6a | 0.0^{E}c |
| | | (0.0)^{C}c | 16.3^{D}c | | | |
| B | K4179 | 6/6 | 204.05 ± | 6/6b | 6/6b | 3.3 ± |
| | | (3.5)a | 43.2a | | | 0.7a |
| C | K4110A | 6/6 | 152.52 ± | 6/6b | 6/6b | 1.8 ± |
| | | (2.0)b | 37.5ab | | | 0.6b |
| D | K5058E | 6/6 | 172.57 ± | 6/6b | 6/6b | 3.4 ± |
| | | (3.8)a | 22ab | | | 0.37a |
| E | K5792A | 6/6 | 133.49 ± | 6/6b | 6/6b | 1.9 ± |
| | | (3.7)a | 27.8bc | | | 1b |
| F | K6836A | 6/6 | 200.59 ± | 6/6b | 6/6b | 2.8 ± |
| | | (3.7)a | 39.4a | | | 0.7ab |
| G | R strain | 6/6 | 132.11 ± | 6/6b | 6/6b | 2.4 ± |
| | | (3.7)a | 23.5bc | | | 0.5ab |

^{A}Values within a column at a specific time point with different lowercase letters are significantly different (P ≤ 0.05)
^{B}No. of positive samples/No. of tested samples (Air sac score ≥ 1)
^{C}Mean score (Macroscopically scored from 0 to 4)
^{D}Mean thickness for the group ± SD (μm)
^{E}Mean (genome) copy number log10 ± SD

TABLE 13
Trial 2 (Turkey Safety 2). Clinical signs in turkeys
at two weeks post inoculation with K4179, K4110A,
K5058E, K5792A, K6836A or R strain.

| | Vaccine/ | |
|---|---|---|
| Group | Challenge | Clinical Picture |
| A | None | No respiratory clinical signs observed |
| B | K4179 | Rales |
| C | K4110A | No respiratory clinical signs observed |
| D | K5058E | Gasping, rales and coughing |
| E | K5792A | Gasping, rales and coughing + flaccid |
| | | enlarged hearts |
| F | K6836A | No respiratory clinical signs observed |
| G | R strain | Weak rales |

TABLE 14
Trial 3 (Chicken Safety and Efficacy). MG serological
response, of chickens 14 days post inoculation with
K6067, K4110A or R strain (Necropsy 1).^{A}

| | Vaccine/ | | | | | | |
|---|---|---|---|---|---|---|---|
| Group | Challenge | SPA | | HI | | ELISA | |
| A | None | 0/9 ^{B} | (0.0)^{C}c | 0/9 | (0.0)^{D}b | 0/9 | (0.0)^{E}b |
| B | K6067 | 10/10 | (1.8)b | 1/10 | (0.1)b | 1/10 | (0.1)ab |
| C | K4110A | 10/10 | (1.5)b | 3/10 | (0.3)ab | 1/10 | (0.1)ab |
| D | R strain | 11/11 | (2.6)a | 7/11 | (0.9)a | 4/11 | (1.3)a |

^{A}Values within a column at a specific time point with different lowercase letters are significantly different (P ≤ 0.05)
^{B} No. of positive samples/No. of tested samples (SPA: ≥1, HI: ≥20, ELISA: ≥0.5, Air sac score ≥ 1)
^{C}Mean agglutination grade (from 0 to 4).
^{D}Mean titer log10
^{E}Mean sample/positive ratio

TABLE 15
Trial 3 (Chicken Safety and Efficacy). Lesion scores, MG
isolation and qPCR from chickens at 14 days post inoculation
with vaccine candidates or R-strain (Necropsy 1).^{A}

| | Vaccine/ | Air sac | Tracheal mucosal | MG isolation | | |
|---|---|---|---|---|---|---|
| Group | Challenge | lesion score | thickness | Trachea | Air sacs | qPCR − MG |
| A | None | 0/9^{B}(0.0)^{C}b | 74 ± 20.5^{D}a | 0/9a | 0/9a | 0.0^{E}b |
| B | K6067 | 0/10 (0.0)b | 88.8 ± 32.1a | 10/10b | 0/10a | 1.5 ± 0.8a |
| C | K4110A | 0/10 (0.0)b | 76.9 ± 14.6a | 10/10b | 0/10a | 0.5 ± 0.5b |
| D | R strain | 9/11 (2.0)a | 136.3 ± 97.4a | 11/11b | 11/11b | 1.9 ± 0.7a |

^{A}Values within a column at a specific time point with different lowercase letters are significantly different (P ≤ 0.05)
^{B}No. of positive samples/No. of tested samples (Air sac score ≥ 1)
^{C}Mean score (Macroscopically scored from 0 to 4)
^{D}Mean thickness for the group ± SD (μm)
^{E}Mean (genome) copy number log10 ± SD

TABLE 16
Trial 3 (Chicken Safety and Efficacy). MG serological
response, of chickens 19 days post mingling with vaccinated
groups (groups B & C) (Necropsy 2).^{A}

| Group | Vaccine/Challenge | SPA | | HI | | ELISA | |
|---|---|---|---|---|---|---|---|
| B | K6067 | 1/5^{B} | (0.2^{C})a | 0/5 | (0^{D})a | 0/5 | (0^{E})a |
| C | K4110A | 1/5 | (0.2)a | 0/5 | (0)a | 0/5 | (0)a |

^{A}Values within a column at a specific time point with different lowercase letters are significantly different (P ≤ 0.05)
^{B}No. of positive samples/No. of tested samples (SPA: ≥1, HI: ≥20, ELISA: ≥0.5, Air sac score ≥ 1)
^{C}Mean agglutination grade (from 0 to 4).
^{D}Mean titer log10
^{E}Mean sample/positive ratio

TABLE 17
Trial 3 (Chicken Safety and Efficacy). Lesion scores, MG
isolation and qPCR from chickens 19 days post mingling with
vaccinated groups (groups B & C) (Necropsy 2).^{A}

| | | Air sac | Tracheal | | | |
|---|---|---|---|---|---|---|
| | Vaccine/ | lesion | mucosal | MG isolation | | qPCR − |
| Group | Challenge | score | thickness | Trachea | Air sacs | MG |
| B | K6067 | 0/5^{B} | 70.9 ± | 2/5a | 0/5a | 0.8 ± |
| | | (0.0^{C})a | 14.^{4D}a | | | 1.^{3E}a 3E |
| C | K4110A | 0/5 | 61 ± | 1/5a | 0/5a | 0.1 ± |
| | | (0.0)a | 19.4a | | | 0.2a |

^{A}Values within a column at a specific time point with different lowercase letters are significantly different (P ≤ 0.05)
^{B}No. of positive samples/No. of tested samples (Air sac score ≥ 1; trachea MG isolation; airsacs MG isolation)
^{C}Mean score (Macroscopically scored from 0 to 4)
^{D}Mean thickness for the group ± SD (μm)
^{E}Mean (genome) copy number log10 ± SD

TABLE 18
Trial 3 (Chicken Safety and Efficacy). MG serological response,
of chickens 28 days post inoculation with vaccine candidates or
R strain and 14 days post challenge with R strain (Necropsy 3).^{A}

| Group | Vaccine | Challenge | SPA | | HI | | ELISA | |
|---|---|---|---|---|---|---|---|---|
| A | None | None | 0/9 ^{B} | (0.0)^{C}b | 0/9 | (0.0)^{D}c | 0/9 | (0.0)^{E}c |
| B-1 | K6067 | Aerosol | 10/10 | (3.4)a | 9/10 | (1.4)ab | 5/10 | (0.8)bc |
| B-2 | K6067 | Contact | 13/13 | (3.4)a | 13/13 | (1.5)ab | 11/13 | (0.7)bc |
| C-1 | K4110A | Aerosol | 10/10 | (3.9)a | 10/10 | (1.6)a | 10/10 | (1.8)a |
| C-2 | K4110A | Contact | 12/13 | (3.1)a | 12/13 | (1.4)ab | 12/13 | (0.7)bc |
| D-1 | None | Aerosol | 10/10 | (3.5)a | 7/10 | (1.1)b | 4/10 | (0.9)b |
| D-2 | None | Contact | 1/10 | (0.1)a | 0/10 | (0)c | 0/10 | (0)c |

^{A}Values within a column at a specific time point with different lowercase letters are significantly different (P ≤ 0.05)
^{B} No. of positive samples/No. of tested samples (SPA: ≥1, HI: ≥20, ELISA: ≥0.5, Air sac score ≥ 1)
^{C}Mean agglutination grade (from 0 to 4).
^{D}Mean titer log10
^{E}Mean sample/positive ratio

TABLE 19
Trial 3 (Chicken Safety and Efficacy). Lesion scores and MG isolation
results 28 days post inoculation with vaccine candidates or R strain
and 14 days post challenge with R strain (Necropsy 3).^{A}

| | | | | | MG isolation | |
|---|---|---|---|---|---|---|
| Group | Vaccine | Challenge | Air sac lesion score | | Trachea | Air sacs |
| A | None | None | 0/9^{B} | (0.0)^{C}b | 0/9c | 0/9b |
| B-1 | K6067 | Aerosol | 1/10 | (0.1)b | 10/10a | 3/10ab |
| B-2 | K6067 | Contact | 0/13 | (0)b | 13/13a | 1/13b |
| C-1 | K4110A | Aerosol | 3/10 | (0.3)b | 10/10a | 3/10ab |
| C-2 | K4110A | Contact | 0/13 | (0)b | 13/13a | 2/13b |
| D-1 | None | Aerosol | 8/10 | (2.0)a | 10/10a | 7/10a |
| D-2 | None | Contact | 2/10 | (0.2)b | 7/10b | 2/10ab |

^{A}Values within a column at a specific time point with different lowercase letters are significantly different (P ≤ 0.05)
^{B}No. of positive samples/No. of tested samples (Air sac score ≥ 1; No. of MG isolated samples/No. of tested samples)
^{C}Mean score (Macroscopically scored from 0 to 4)

TABLE 20
Trial 3 (Chicken Safety and Efficacy). MG, R-strain specific,
K6067 specific and K4110A specific qPCR from chickens 28
days post inoculation with vaccine candidates or R strain and
14 days post challenge with R strain (Necropsy 3).^{A}

| | | Challenge | qPCR − | qPCR − | qPCR − | qPCR − |
|---|---|---|---|---|---|---|
| Group | Vaccine | (R-strain) | MG | K6067 | K4110A | R |
| A | None | None | 0.0^{B}b | 0.0a | 0.0a | 0.0bc |
| B-1 | K6067 | Aerosol | 2.6 ± | 2.5 ± | N/A | 0.2 ± |
| | | | 0.3a | 0.3b | | 0.4bc |
| B-2 | K6067 | Contact | 2.6 ± | 2.4 ± | N/A | 0.0c |
| | | | 0.4a | 0.4b | | |
| C-1 | K4110A | Aerosol | 2.2 ± | N/A | 1.5 ± | 0.9 ± |
| | | | 0.9a | | 1.1b | 0.9b |
| C-2 | K4110A | Contact | 2.1 ± | N/A | 2.0 ± | 0.0c |
| | | | 0.9a | | 1.0 b | |
| D-1 | None | Aerosol | 2.5 ± | 0.0a | 0.0a | 2.8 ± |
| | | | 1.3a | | | 1.3a |
| D-2 | None | Contact | 0.5 ± | 0.0a | 0.0a | 0.4 ± |
| | | | 0.8b | | | 1.0bc |

A Values within a column at a specific time point with different lowercase letters are significantly different (P ≤ 0.05)
B Mean (genome) copy number log10 ± SD

TABLE 21
Trial 3 (Chicken Safety and Efficacy). Tracheal mucosal thickness
of chickens 28 days post inoculation with vaccine candidates
or R strain and 14 days post challenge with R strain or mingling
with R strain-challenged chickens (Necropsy 3).^{A}

| Group | Vaccine/Challenge | Tracheal mucosal thickness |
|---|---|---|
| A | None | 82.31 ± 14.64^{B}a |
| B | K6067 | 80.43 ± 11.86a |
| C | K4110A | 85.53 ± 17.73a |
| D | R strain | 123.15 ± 106.44a |

^{A}Values within a column at a specific time point with different lowercase letters are significantly different (P ≤ 0.05)
^{B}Mean thickness for the group ± SD (μm)

TABLE 22
Trial 4 (Dose Response 1). MG isolation and qPCR from chickens
at 7 days post inoculation with K6067 (1^{st} sampling).^{A}

| | | MG isolation | qPCR − MG |
|---|---|---|---|
| Group | Inoculation | (choanal cleft) | (trachea) |
| A | None | 0/7^{B}b | 0.0^{C}b |
| B | None | 0/7b | 0.0b |
| C | 10¹ CCU/mL eye drop K6067 | 0/7b | 0.0b |
| D | 10² CCU/mL eye drop K6067 | 1/7b | 0.0b |
| E | 10³ CCU/mL eye drop K6067 | 7/7a | 0.16 ± 0.42b |
| F | 10⁴ CCU/mL eye drop K6067 | 7/7a | 0.23 ± 0.62ab |
| G | 10⁵ CCU/mL eye drop K6067 | 7/7a | 0.84 ± 0.92a |
| H | 10³ CCU/mL aerosol K6067 | 0/7b | 0.0b |
| I | 10⁴ CCU/mL aerosol K6067 | 0/7b | 0.0b |
| J | 10⁵ CCU/mL aerosol K6067 | 1/7b | 0.0b |
| K | 10⁶ CCU/mL aerosol K6067 | 1/7b | 0.0b |
| L | 10⁷ CCU/mL aerosol K6067 | 0/7b | 0.0b |

^{A}Values within a column at a specific time point with different lowercase letters are significantly different (P ≤ 0.05)
^{B}No. of positive samples/No. of tested samples (color changing as the result of Mycoplasm growth)
^{C}Mean (genome) copy number log10 ± SD

TABLE 23
Trial 4 (Dose Response 1). MG serological response, of chickens
21 days post inoculation with K6067 (2^{nd} sampling).^{A}

| Group | Inoculation | SPA | | HI | | ELISA | |
|---|---|---|---|---|---|---|---|
| A | None | 0/7 ^{B} | (0.0)^{C}c | 0/7 | (0.0)^{D}a | 0/7 | (0.0)^{E}b |
| B | None | 0/7 | (0.0)c | 0/7 | (0.0)a | 0/7 | (0.0)b |
| C | 10¹ CCU/mL eye drop K6067 | 0/7 | (0.0)c | 0/7 | (0.0)a | 0/7 | (0.0)b |
| D | 10² CCU/mL eye drop K6067 | 2/7 | (0.5)bc | 0/7 | (0.0)a | 0/7 | (0.0)b |
| E | 10³ CCU/mL eye drop K6067 | 6/7 | (1.1)ab | 0/7 | (0.0)a | 0/7 | (0.1)b |
| F | 10⁴ CCU/mL eye drop K6067 | 6/7 | (1.0)ab | 1/7 | (0.1)a | 0/7 | (0.1)b |
| G | 10⁵ CCU/mL eye drop K6067 | 6/7 | (1.4)a | 0/7 | (0.0)a | 2/7 | (0.3)a |
| H | 10³ CCU/mL aerosol K6067 | 0/7 | (0.0)c | 0/7 | (0.0)a | 0/7 | (0.0)b |
| I | 10⁴ CCU/mL aerosol K6067 | 0/7 | (0.0)c | 0/7 | (0.0)a | 0/7 | (0.0)b |
| J | 10⁵ CCU/mL aerosol K6067 | 1/7 | (0.1)c | 0/7 | (0.0)a | 0/7 | (0.0)b |
| K | 10⁶ CCU/mL aerosol K6067 | 1/7 | (0.1)c | 0/7 | (0.0)a | 0/7 | (0.0)b |
| L | 10⁷ CCU/mL aerosol K6067 | 0/7 | (0.0)c | 0/7 | (0.0)a | 0/7 | (0.0)b |

^{A}Values within a column at a specific time point with different lowercase letters are significantly different (P ≤ 0.05)
^{B} No. of positive samples/No. of tested samples (SPA: ≥1, HI: ≥20, ELISA: ≥0.5, Air sac score ≥ 1)
^{C}Mean agglutination grade (from 0 to 4).
^{D}Mean titer log10
^{E}Mean sample/positive ratio

TABLE 24
Trial 4 (Dose Response 1). MG isolation and qPCR from chickens
at 21 days post inoculation with K6067 (2^{nd} sampling).^{A}

| | | MG isolation | |
|---|---|---|---|
| | | (choanal | qPCR − MG |
| Group | Inoculation | cleft) | (trachea) |
| A | None | 0/7^{B}b | 0.0^{C}c |
| B | None | 0/7b | 0.0c |
| C | 10¹ CCU/mL eye drop K6067 | 0/7b | 0.0c |
| D | 10² CCU/mL eye drop K6067 | 2/7b | 0.32 ± 0.61bc |
| E | 10³ CCU/mL eye drop K6067 | 7/7a | 0.45 ± 0.59abc |
| F | 10⁴ CCU/mL eye drop K6067 | 7/7a | 1.00 ± 0.58a |
| G | 10⁵ CCU/mL eye drop K6067 | 7/7a | 0.92 ± 0.66ab |
| H | 10³ CCU/mL aerosol K6067 | 0/7b | 0.0c |
| I | 10⁴ CCU/mL aerosol K6067 | 0/7b | 0.0c |
| J | 10⁵ CCU/mL acrosol K6067 | 0/7b | 0.0c |
| K | 10⁶ CCU/mL aerosol K6067 | 1/7b | 0.0c |
| L | 10⁷ CCU/mL aerosol K6067 | 0/7b | 0.0c |

^{A}Values within a column at a specific time point with different lowercase letters are significantly different (P ≤ 0.05)
^{B}No. of positive samples/No. of tested samples (color changing as the result of Mycoplasm growth)
^{C}Mean (genome) copy number log10 ± SD

TABLE 25
Trial 4 (Dose Response 1). MG serological response, of chickens 6 weeks post
inoculation with K6067 and 2 weeks post challenge with R strain (necropsy).^{A}

| Group | Inoculation | SPA | | HI | | ELISA | |
|---|---|---|---|---|---|---|---|
| A | None | 0/7 ^{B} | (0.0)^{C}b | 0/7 | (0.0)^{D}c | 0/7 | (0.0)^{E}d |
| B | R strain only | 7/7 | (2.2)a | 7/7 | (1.6)ab | 7/7 | (1.7)abc |
| C | 10¹ CCU/mL eye drop K6067 + R strain | 7/7 | (2.1)a | 7/7 | (1.8)a | 7/7 | (1.8)bcd |
| D | 10² CCU/mL eye drop K6067 + R strain | 7/7 | (2.2)a | 7/7 | (1.8)ab | 7/7 | (1.9)abc |
| E | 10³ CCU/mL eye drop K6067 \| R strain | 7/7 | (2.0)a | 7/7 | (1.8)a | 7/7 | (2.3)abc |
| F | 10⁴ CCU/mL eye drop K6067 + R strain | 7/7 | (1.7)a | 7/7 | (1.9)a | 7/7 | (2.7)ab |
| G | 10⁵ CCU/mL eye drop K6067 + R strain | 7/7 | (1.8)a | 7/7 | (1.5)ab | 7/7 | (2.8)a |
| H | 10³ CCU/mL aerosol K6067 + R strain | 7/7 | (2.0)a | 7/7 | (1.5)ab | 6/7 | (1.4)abcd |
| I | 10⁴ CCU/mL aerosol K6067 + R strain | 7/7 | (1.5)a | 7/7 | (1.6)ab | 7/7 | (1.3)abcd |
| J | 10⁵ CCU/mL aerosol K6067 + R strain | 4/7 | (1.2)a | 5/7 | (1.2)b | 4/7 | (0.8)cd |
| K | 10⁶ CCU/mL aerosol K6067 + R strain | 7/7 | (2.2)a | 7/7 | (1.6)ab | 7/7 | (1.2)bcd |
| L | 10⁷ CCU/mL aerosol K6067 + R strain | 7/7 | (2.0)a | 7/7 | (1.9)a | 7/7 | (1.3)abcd |

^{A}Values within a column at a specific time point with different lowercase letters are significantly different (P ≤ 0.05)
^{B} No. of positive samples/No. of tested samples (SPA: ≥1, HI: ≥20, ELISA: ≥0.5, Air sac score ≥ 1)
^{C}Mean agglutination grade (from 0 to 4).
^{D}Mean titer log10
^{E}Mean sample/positive ratio

TABLE 26
Trial 4 (Dose Response 1). Lesion scores and MG isolation results 6 weeks post
inoculation with K6067 and 2 weeks post challenge with R strain (necropsy).^{A}

| | | | | MG isolation | |
|---|---|---|---|---|---|
| | | Air sac | Tracheal mucosal | Choanal | Air |
| Group | Inoculation | lesion score | thickness | cleft | sacs |
| A | None | 0/7^{B} (0.0^{C})d | 92.4 ± 18.5^{D}bc | 0/7^{E}c | 0/7c |
| B | R strain only | 7/7 (1.9)abd | 236.5 ± 101.2abc | 7/7a | 7/7a |
| C | 10¹ CCU/mL eye drop K6067 + R strain | 7/7 (2.1)ab | 296.1 ± 152.1ab | 7/7a | 7/7a |
| D | 10² CCU/mL eye drop K6067 + R strain | 3/7 (0.7)abcd | 219.9 ± 200.8abc | 7/7a | 7/7a |
| E | 10³ CCU/mL eye drop K6067 + R strain | 2/7 (0.3)bcd | 75.0 ± 18.3bc | 7/7a | 3/7b |
| F | 10⁴ CCU/mL eye drop K6067 + R strain | 2/7 (0.3)bcd | 66.7 ± 14.6c | 7/7a | 5/7ab |
| G | 10⁵ CCU/mL eye drop K6067 + R strain | 1/7 (0.1)cd | 80.0 ± 20.2bc | 7/7a | 7/7a |
| H | 10³ CCU/mL aerosol K6067 + R strain | 6/7 (2.4)a | 248.7 ± 137.5abc | 7/7a | 7/7a |
| I | 10⁴ CCU/mL aerosol K6067 + R strain | 7/7 (1.5)a | 257.0 ± 151.6abc | 7/7a | 7/7a |
| J | 10⁵ CCU/mL aerosol K6067 + R strain | 7/7 (2.0)abc | 180.2 ± 150.6abc | 5/7b | 7/7a |
| K | 10⁶ CCU/mL aerosol K6067 + R strain | 4/7 (1.0)abcd | 195.9 ± 144.6abc | 7/7a | 7/7a |
| L | 10⁷ CCU/mL aerosol K6067 + R strain | 6/7 (2.0)abc | 405.5 ± 163.5a | 7/7a | 7/7a |

^{A}Values within a column at a specific time point with different lowercase letters are significantly different (P ≤ 0.05)
^{B}No. of positive samples/No. of tested samples (Air sac score ≥ 1)
^{C}Mean score (Macroscopically scored from 0 to 4)
^{D}Mean thickness for the group ± SD (μm)
^{E}No. of positive samples/No. of tested samples (culture)

TABLE 27
Trial 4 (Dose Response 1). Lesion scores and MG isolation results 6 weeks post
inoculation with K6067 and 2 weeks post challenge with R strain (necropsy).^{A}

| | | | | MG isolation | |
|---|---|---|---|---|---|
| | | Air sac | Tracheal mucosal | Choanal | Air |
| Group | Inoculation | lesion score | thickness | cleft | sacs |
| A | None | 0/7^{B} (0.0^{C})d | 92.4 ± 18.5^{D}bc | 0/7^{E}c | 0/7c |
| B | R strain only | 7/7 (1.9)abd | 236.5 ± 101.2abc | 7/7a | 7/7a |
| C | 10¹ CCU/mL eye drop K6067 + R strain | 7/7 (2.1)ab | 296.1 ± 152.1ab | 7/7a | 7/7a |
| D | 10² CCU/mL eye drop K6067 + R strain | 3/7 (0.7)abcd | 219.9 ± 200.8abc | 7/7a | 7/7a |
| E | 10³ CCU/mL eye drop K6067 + R strain | 2/7 (0.3)bcd | 75.0 ± 18.3bc | 7/7a | 3/7b |
| F | 10⁴ CCU/mL eye drop K6067 + R strain | 2/7 (0.3)bcd | 66.7 ± 14.6c | 7/7a | 5/7ab |
| G | 10⁵ CCU/mL eye drop K6067 + R strain | 1/7 (0.1)cd | 80.0 ± 20.2bc | 7/7a | 7/7a |
| H | 10³ CCU/mL aerosol K6067 + R strain | 6/7 (2.4)a | 248.7 ± 137.5abc | 7/7a | 7/7a |
| I | 10⁴ CCU/mL aerosol K6067 + R strain | 7/7 (1.5)a | 257.0 ± 151.6abc | 7/7a | 7/7a |
| J | 10⁵ CCU/mL aerosol K6067 + R strain | 7/7 (2.0)abc | 180.2 ± 150.6abc | 5/7b | 7/7a |
| K | 10⁶ CCU/mL aerosol K6067 + R strain | 4/7 (1.0)abcd | 195.9 ± 144.6abc | 7/7a | 7/7a |
| L | 10⁷ CCU/mL aerosol K6067 + R strain | 6/7 (2.0)abc | 405.5 ± 163.5a | 7/7a | 7/7a |

^{A}Values within a column at a specific time point with different lowercase letters are significantly different (P ≤ 0.05)
^{B}No. of positive samples/No. of tested samples (Air sac score ≥ 1)
^{C}Mean score (Macroscopically scored from 0 to 4)
^{D}Mean thickness for the group ± SD (μm)
^{E}No. of positive samples/No. of tested samples (culture)

TABLE 28
Trial 4 (Dose Response 1). MG, R-strain specific and K6067 specific genome copy numbers 6 weeks
post inoculation with vaccine candidate and 2 weeks post challenge with R strains (necropsy).^{A}

| | | | | qPCR - |
|---|---|---|---|---|
| Group | Inoculation | qPCR - MG | qPCR - R strain | K6067 |
| A | None | 0.0^{B}f | 0.0c | 0.0a |
| B | R strain only | 3.09 ± 0.45ab | 3.56 ± 0.47a | 0.0a |
| C | 10¹ CCU/mL eye drop K6067 + R strain | 3.15 ± 0.62ab | 3.41 ± 0.73ab | 0.0a |
| D | 10² CCU/mL eye drop K6067 + R strain | 2.59 ± 1.33abcd | 2.62 ± 1.34ab | 0.40 ± 1.06a |
| E | 10³ CCU/mL eye drop K6067 + R strain | 0.66 ± 0.65ef | 0.06 ± 0.16c | 0.19 ± 0.46a |
| F | 10⁴ CCU/mL eye drop K6067 + R strain | 1.26 ± 0.86def | 0.39 ± 0.80c | 0.79 ± 0.96a |
| G | 10⁵ CCU/mL eye drop K6067 + R strain | 1.53 ± 0.75cde | 0.43 ± 0.68c | 0.85 ± 0.87a |
| H | 10³ CCU/mL aerosol K6067 + R strain | 3.12 ± 0.44ab | 3.42 ± 0.46ab | 0.0a |
| I | 10⁴ CCU/mL aerosol K6067 + R strain | 3.31 ± 0.92ab | 3.44 ± 0.68ab | 0.0a |
| J | 10⁵ CCU/mL aerosol K6067 + R strain | 1.95 ± 1.25bcde | 2.11 ± 1.54b | 0.0a |
| K | 10⁶ CCU/mL aerosol K6067 + R strain | 2.73 ± 0.66abc | 3.06 ± 0.81ab | 0.24 ± 0.63a |
| L | 10⁷ CCU/mL aerosol K6067 + R strain | 3.48 ± 0.33a | 3.90 ± 0.24a | 0.20 ± 0.53a |

^{A}Values within a column at a specific time point with different lowercase letters are significantly different (P ≤ 0.05)
^{B}Mean (genome) copy number log10 ± SD

TABLE 29
Trial 4 (Dose Response 1). Minimum infective dose 50% (MID50) of K6067 based
on qPCR and Mycoplasma culture results of 1^{st} and 2^{nd} sampling.

| | Parameter | Timing | MID50 | | Descriptions |
|---|---|---|---|---|---|
| 1 | qPCR results of groups C-G^{A} | 1^{st} | N/A | | None of the groups were |
| | | sampling^{B} | | | infected at 50% or higher |
| 2 | qPCR results of groups H-L^{C} | 1^{st} | N/A | | None of the groups were |
| | | sampling | | | infected at 50% or higher |
| 3 | Mycoplasma culture results | 1^{st} | 10^{3.8} | CCU/mL | Group E and F were infected |
| | of groups C-G | sampling | | | 38 and 53%, respectively |
| 4 | Mycoplasma Culture results | 1^{st} | N/A | | None of the groups were |
| | of groups H-L | sampling | | | infected at 50% or higher |
| 5 | qPCR results of groups C-G | 2^{nd} | N/A | | None of the groups were |
| | | sampling^{D} | | | infected at 50% or higher |
| 6 | qPCR results of groups H-L | 2^{nd} | N/A | | None of the groups were |
| | | sampling | | | infected at 50% or higher |
| 7 | Mycoplasma Culture results | 2^{nd} | 10^{3.53} | CCU/mL | Group E and F were infected |

| | | | | |
|---|---|---|---|---|
| | of groups C-G | sampling | | 42 and 57%, respectively |
| 8 | Mycoplasma Culture results | 2^{nd} | N/A | None of the groups were |
| | of groups H-L | sampling | | infected at 50% or higher |

^{A}Eye drop inoculated groups
^{B}1st sampling was performed 1 week post inoculation at 4 WOA.
^{C}Aerosol-inoculated groups
^{D}2nd sampling was performed 3 weeks post inoculation at 6 WOA.

TABLE 30
Trial 4 (Dose Response 1). Minimum protective dose
50% (MPD50) of K6067 based on air sac lesion scores
and tracheal thickness measurements at necropsy.

| No. | Parameter | MPD50 | Descriptions |
|---|---|---|---|
| 1 | Airsac scores of | 10⁴ | Group F was 50% protected |
| | groups C-G^{A} | | cumulatively. |
| 2 | Airsac scores of | N/A | None of the groups were |
| | groups H-L^{B} | | infected at 50% or higher |
| 3 | Tracheal thickness | 10^{3.3} | Group E and F were protected |
| | measurements^{C} | CCU/mL | at 47 and 60%, respectively |
| | of groups C-G | | |
| 4 | Tracheal thickness | N/A | None of the groups were |
| | measurements | | infected at 50% or higher |
| | of groups H-L | | |

^{A}Eye drop inoculated groups
^{B}Aerosol-inoculated groups
^{C}To compare the tracheal thickness measurements and consider the measurements as affected and unaffected, the highest measurement in group A (negative control), which was 118.2 μm, was considered as the threshold.

TABLE 31
Trial 5 (Dose Response 2). MG isolation and qPCR from chickens
at 7 days post inoculation with K4110A (1^{st} sampling).^{A}

| | | MG isolation | qPCR - MG |
|---|---|---|---|
| Group | Inoculation | (choanal cleft) | (trachea) |
| A | None | 0/7^{B}c | 0.0^{C}c |
| B | None | 0/7c | 0.0c |
| C | 10³ CCU/mL eye | 10/13b | 0.18 ± 0.65c |
| | drop K4110A | | |
| D | 10⁴ CCU/mL eye | 13/13a | 0.98 ± 1.42bc |
| | drop K4110A | | |
| E | 10⁵ CCU/mL eye | 13/13a | 1.23 ± 0.93abc |
| | drop K4110A | | |
| F | 10⁶ CCU/mL eye | 13/13a | 1.72 ± 1.14ab |
| | drop K4110A | | |
| G | 10⁷ CCU/mL eye | 13/13a | 2.22 ± 1.01a |
| | drop K4110A | | |

^{A}Values within a column at a specific time point with different lowercase letters are significantly different (P ≤ 0.05)
^{B}No. of positive samples/No. of tested samples (color changing as the result of Mycoplasm growth)
^{C}Mean (genome) copy number log10 ± SD

TABLE 32
Trial 5 (Dose Response 2). MG serological response, of chickens
21 days post inoculation with K4110A (2^{nd} sampling).^{A}

| Group | Inoculation | SPA | HI | ELISA |
|---|---|---|---|---|
| A | None | 0/7 ^{B} (0.0)^{C}b | 0/7 (0.0)^{D}b | 0/7 (0.0)^{E}ab |
| B | None | 0/7 (0.0)b | 0/7 (0.0)b | 0/7 (0.0)ab |
| C | 10³ CCU/mL eye drop K4110A | 4/13 (0.3)b | 0/13 (0.0)b | 0/13 (0.0)b |
| D | 10⁴ CCU/mL eye drop K4110A | 13/13 (1.3)a | 4/13 (0.4)ab | 0/13 (0.0)b |
| E | 10⁵ CCU/mL eye drop K4110A | 12/13 (1.7)a | 4/13 (0.4)ab | 2/13 (0.1)ab |
| F | 10⁶ CCU/mL eye drop K4110A | 13/13 (1.4)a | 2/13 (0.2)b | 0/13 (0.0)ab |
| G | 10⁷ CCU/mL eye drop K4110A | 13/13 (1.5)a | 10/13 (1.1)a | 2/13 (0.2)a |

^{A}Values within a column at a specific time point with different lowercase letters are significantly different (P ≤ 0.05)
^{B} No. of positive samples/No. of tested samples (SPA: ≥1, HI: ≥20, ELISA: ≥0.5, Air sac score ≥1)
^{C}Mean agglutination grade (from 0 to 4).
^{D}Mean titer log10
^{E}Mean sample/positive ratio

TABLE 33
Trial 5 (Dose Response 2). MG isolation and qPCR from chickens
at 21 days post inoculation with K4110A (2^{nd} sampling).^{A}

| | | MG isolation | qPCR - MG |
|---|---|---|---|
| Group | Inoculation | (choanal cleft) | (trachea) |
| A | None | 0/7^{B}b | 0.0^{C}c |
| B | None | 0/7b | 0.0c |
| C | 10³ CCU/mL eye | 11/13a | 0.53 ± 0.92bc |
| | drop K4110A | | |
| D | 10⁴ CCU/mL eye | 13/13a | 0.77 ± 1.31bc |
| | drop K4110A | | |
| E | 10⁵ CCU/mL eye | 13/13a | 1.62 ± 1.18ab |
| | drop K4110A | | |
| F | 10⁶ CCU/mL eye | 13/13a | 1.33 ± 1.00abc |
| | drop K4110A | | |
| G | 10⁷ CCU/mL eye | 13/13a | 2.13 ± 0.81a |
| | drop K4110A | | |

^{A}Values within a column at a specific time point with different lowercase letters are significantly different (P ≤ 0.05)
^{B}No. of positive samples/No. of tested samples (color changing as the result of Mycoplasm growth)
^{C}Mean (genome) copy number log10 ± SD

TABLE 34
Trial 5 (Dose Response 2). MG serological response, of chickens 6 weeks post
inoculation with K4110A and 2 weeks post challenge with R strain (necropsy).^{A}

| Group | Inoculation | SPA | HI | ELISA |
|---|---|---|---|---|
| A | None | 0/7 ^{B} (0.0)^{C}c | 0/7 (0.0)^{D}c | 0/7 (0.0)^{E}c |
| B | R strain only | 7/7 (2.4)a | 6/7 (1.3)b | 6/7 (0.8)bc |
| C | 10³ CCU/mL eye drop K4110A + R strain | 12/12 (1.7)ab | 12/12 (1.8)a | 12/12 (1.7)a |
| D | 10⁴ CCU/mL eye drop K4110A + R strain | 13/13 (1.5)b | 13/13 (1.9)a | 13/13 (1.4)ab |
| E | 10⁵ CCU/mL eye drop K4110A + R strain | 13/13 (1.6)ab | 13/13 (1.9)a | 13/13 (1.5)ab |
| F | 10⁶ CCU/mL eye drop K4110A + R strain | 13/13 (1.7)ab | 13/13 (1.9)a | 13/13 (1.2)ab |
| G | 10⁷ CCU/mL eye drop K4110A + R strain | 13/13 (1.8)ab | 13/13 (2.0)a | 13/13 (1.7)a |

^{A}Values within a column at a specific time point with different lowercase letters are significantly different (P ≤ 0.05)
^{B} No. of positive samples/No. of tested samples (SPA: ≥1, HI: ≥20, ELISA: ≥0.5, Air sac score ≥1)
^{C}Mean agglutination grade (from 0 to 4).
^{D}Mean titer log10
^{E}Mean sample/positive ratio

TABLE 35
Trial 5 (Dose Response 2). Lesion scores, MG isolation and qPCR results 6 weeks post
inoculation with K4110A and 2 weeks post challenge with R strain (necropsy).^{A}

| | | | | MG isolation | |
|---|---|---|---|---|---|
| | | Air sac | Tracheal mucosal | Choanal | Air |
| Group | Inoculation | lesion score | thickness | cleft | sacs |
| A | None | 0/7^{B} (0.0^{C})c | 71.1 ± 11.2^{D}b | 0/7^{E}a | 0/7^{E}a |
| B | R strain only | 6/7 (2.0)a | 224.1 ± 196.7a | 7/7b | 7/7b |
| C | 10³ CCU/mL eye drop K4110A + R strain | 8/12 (0.8)b | 76.4 ± 17.9b | 12/12b | 12/12b |
| D | 10⁴ CCU/mL eye drop K4110A + R strain | 3/13 (0.2)bc | 73.9 ± 10.5b | 13/13b | 11/13b |
| E | 10⁵ CCU/mL eye drop K4110A + R strain | 4/13 (0.3)bc | 66.8 ± 15.7b | 13/13b | 13/13b |
| F | 10⁶ CCU/mL eye drop K4110A + R strain | 0/13 (0.0)c | 68.9 ± 19.8b | 13/13b | 12/13b |
| G | 10⁷ CCU/mL eye drop K4110A + R strain | 3/13 (0.2)bc | 68.0 ± 16.1b | 13/13b | 13/13b |

^{A}Values within a column at a specific time point with different lowercase letters are significantly different (P ≤ 0.05)
^{B}No. of positive samples/No. of tested samples (Air sac score ≥ 1)
^{C}Mean score (Macroscopically scored from 0 to 4)
^{D}Mean thickness for the group ± SD (μm)
^{E}No. of positive samples/No. of tested samples (culture)

TABLE 36
Trial 5 (Dose Response 2). MG, R-strain specific and K4110A specific qPCR results on tracheal washes and air
sac cultures, 6 weeks post inoculation with K4110A and 2 weeks post challenge with R strains (necropsy).^{A}

| | | Tracheal wash | | | Air Sac Culture | |
|---|---|---|---|---|---|---|
| Group | Inoculation | qPCR − MG | qPCR − R strain | qPCR − K4110A | qPCR − R strain | qPCR − K4110A |
| A | None | 0.0^{B}b | 0.0c | 0.0c | 0.0b | 0.0a |
| B | R strain only | 2.69 ± 1.13a | 2.56 ± 1.01a | 0.0c | 1.16 ± 1.98b | 0.0a |
| C | 10³ CCU/mL eye drop | 2.27 ± 0.86a | 1.31 ± 1.22b | 0.89 ± 0.88bc | 3.98 ± 0.59a | 0.0a |
| | K4110A + R strain | | | | | |
| D | 10⁴ CCU/mL eye drop | 2.31 ± 0.63a | 0.57 ± 0.69bc | 1.55 ± 0.71ab | 4.62 ± 0.82a | 0.0a |
| | K4110A + R strain | | | | | |
| E | 10⁵ CCU/mL eye drop | 1.83 ± 0.70a | 0.53 ± 0.44bc | 1.30 ± 0.88ab | 4.26 ± 0.52a | 0.0a |
| | K4110A + R strain | | | | | |
| F | 10⁶ CCU/mL eye drop | 2.1 ± 1.4a | 0.41 ± 0.55c | 1.43 ± 0.74ab | 4.63 ± 0.74a | 0.0a |
| | K4110A + R strain | | | | | |
| G | 10⁷ CCU/mL eye drop | 2.07 ± 0.57a | 0.51 ± 0.43bc | 1.76 ± 0.59a | 4.32 ± 0.63a | 0.06 ± 0.22a |
| | K4110A + R strain | | | | | |

^{A}Values within a column at a specific time point with different lowercase letters are significantly different (P ≤ 0.05)
^{B}Mean (genome) copy number log10 ± SD

TABLE 37
Trial 5 (Dose Response 2). Minimum infective dose
50% (MID50) of K4110A based on qPCR and Mycoplasma culture
results of 1^{st} and 2^{nd} sampling.

| No. | parameter | Timing | MID50 | Descriptions |
|---|---|---|---|---|
| 1 | qPCR results of | 1^{st} | 10^{5.3} | Groups E and F were |
| | groups C-G^{A} | sampling^{B} | CCU/mL | infected 48 and 55%, |
| | | | | respectively |
| 2 | Mycoplasma | 1^{st} | N/A | None of the groups |
| | culture results | sampling | | were infected at |
| | of groups C-G | | | lower than 50% rate |
| 3 | qPCR results of | 2^{nd} | 10^{5.2} | Groups E and F were |
| | groups C-G | sampling^{C} | CCU/mL | infected 48 and 57%, |
| | | | | respectively |
| 4 | Mycoplasma | 2^{nd} | N/A | None of the groups |
| | Culture results | sampling | | were infected at |
| | of groups C-G | | | lower than 50% rate |

^{A}Eye drop inoculated groups
^{B}1st sampling was performed 1 week post inoculation at 4 WOA.
^{C}2nd sampling was performed 3 weeks post inoculation at 6 WOA.

TABLE 38
Trial 5 (Dose Response 2). Minimum protective dose
50% (MPD50) of K4110A based on airsac scores and
tracheal thickness measurements at necropsy.

| No. | parameter | MPD50 | Descriptions |
|---|---|---|---|
| 1 | Airsac scores of | 10^{3.7} | Groups C and D unaffected |
| | groups C-G^{A} | | airsac rates were 33 and |
| | | | 56%, respectively. |
| 2 | Tracheal thickness | N/A | None of the groups were |
| | measurements | | positive^{B} at 50% or |
| | of groups C-G | | lower rates |

^{A}Eye drop inoculated groups
^{B}To compare the tracheal thickness measurements and consider the measurements as affected and unaffected, the highest measurement in group A (negative control), which was 91.3 μm, was considered as the threshold.

TABLE 39
Trial 6 (Vertical Transmission). MG yolk antibodies of
last set of eggs laid at 31 WOA; 6 weeks post inoculation
with vaccine candidates, F -strain or R-strain.^{A}

| Group | Vaccine/Challenge | HI | ELISA |
|---|---|---|---|
| A | None | 0/7^{B} (0.0)^{C}b | 0/7^{B} (0.0)^{D}c |
| B | K6067 | 0/14 (0.0)b | 0/14 (0.3)bc |
| C | K4110A | 6/12 (0.7)a | 6/12 (0.7)ab |
| D | F strain | 0/12 (0.0)b | 4/12 (0.5)abc |
| E | R strain | 3/12 (0.3)ab | 9/12 (0.9)a |

^{A}Values within a column at a specific time point with different lowercase letters are significantly different (P ≤ 0.05)
^{B}No. of positive samples/No. of tested samples (HI: ≥20, ELISA: ≥0.5)
^{C}Mean titer log10
^{D}Mean sample/positive ratio

TABLE 40
Trial 6 (Vertical Transmission). Fertility rates and hen-week egg
production means from 24-31 WOA and 21-31 WOA, respectively ^{A}.

| | | | Hen-week egg |
|---|---|---|---|
| | | | production |
| Group | Inoculation | Fertility rate^{B} | means^{C} |
| A | Negative control | 83.7 ± 9^{D}ab | 89.4 ± 9.1a |
| B | K6067 | 77.9 ± 5.7b | 88.2 ± 9a |
| C | K4110A | 84.0 ± 4.2ab | 84.5 ± 4.8a |
| D | F strain | 85.3 ± 5.1a | 85.5 ± 3.4a |
| E | R strain | 83.5 ± 7.6ab | 83.9 ± 6.1a |

^{A} Values within a column at a specific time point with different lowercase letters are significantly different (P ≤ 0.05)
^{B}Number of fertile eggs after 7 DOE divided by the total number of incubated eggs times 100
^{C}Total number of eggs laid on the week divided by the total number of hens times 100
^{D}percentage ± SD

TABLE 41
Trial 6 (Vertical Transmission). MG serological responses
of 31 WOA chickens at 6 weeks post inoculation (necropsy).^{A}

| Group | Inoculation | SPA | HI | ELISA |
|---|---|---|---|---|
| A | Negative | 0/16^{B} (0^{C})a | 0/16 (0^{D})c | 0/16 (0^{E})c |
| | control | | | |
| B | K6067 | 31/31 (3)b | 26/31 (1.19)b | 27/31(1.08)b |
| C | K4110A | 32/32 (3.1)b | 31/32 (1.73)a | 31/32 (2.02)a |
| D | F strain | 31/31 (2.8)b | 31/31 (1.64)a | 31/31 (1.89)a |
| E | R strain | 31/31 (3.3)b | 31/31 (1.87)a | 31/31 (2.27)a |

^{A}Values within a column at a specific time point with different lowercase letters are significantly different (P ≤ 0.05)
^{B}No. of positive samples/No. of tested samples (SPA: ≥1, HI: ≥20, ELISA: ≥0.5, Air sac score ≥1)
^{C}Mean agglutination grade (from 0 to 4).
^{D}Mean titer log10
^{E}Mean sample/positive ratio

TABLE 42
Trial 6 (Vertical Transmission). Lesion scores, MG isolation and qPCR from chickens
at 6 weeks post inoculation with K6067, K4110A, F -strain or R-strain (Necropsy).^{A}

| | Vaccine/ | Air sac | Tracheal mucosal | MG isolation | | qPCR − MG | qPCR − MG |
|---|---|---|---|---|---|---|---|
| Group | Challenge | lesion score | thickness | Choanal cleft | Air sac | Trachea | Oviduct |
| A | None | 2/16^{B} (0.1)^{C} | 61.83 ± 17.03^{D}ab | 0/16a | 0/16b | 0.0^{E}b | 0.0^{E}a |
| B | K6067 | 2/31 (0.1) | 75.40 ± 21.37b | 31/31b | 4/31b | 2.54 ± 1.02b | 0.0a |
| C | K4110A | 2/32 (0.1) | 107.9 ± 90.94b | 31/32b | 8/32b | 2.57 ± 0.96ab | 0.0a |
| D | F strain | 4/31 (0.1) | 73.63 ± 20.9b | 30/31b | 6/31b | 1.89 ± 1.02b | 0.0a |
| E | R strain | 10/31 (0.6) | 123.96 ± 100.7a | 31/31b | 19/31a | 2.2 ± 1.3a | 0.0a |

^{A}Values within a column at a specific time point with different lowercase letters are significantly different (P ≤ 0.05)
^{B}No. of positive samples/No. of tested samples (Air sac score ≥ 1)
^{C}Mean score (Macroscopically scored from 0 to 4)
^{D}Mean thickness for the group ± SD (μm)
^{E}Mean (genome) copy number log10 ±

1. An isolated Mycoplasma gallisepticum strain, wherein the isolated Mycoplasma gallisepticum strain comprises the K6067 Mycoplasma gallisepticum strain deposited at the ATCC under Patent Designation PTA-127168, or a progeny or derivative thereof, or comprises the K4110 Mycoplasma gallisepticum strain deposited at the ATCC under Patent Designation PTA-127282, and progeny and derivatives thereof.

2-3. (canceled)

4. A composition comprising the isolated Mycoplasma gallisepticum of claim 1.

5. The composition of claim 4 comprising water.

6. The composition of claim 4 comprising a pharmaceutically acceptable carrier.

7. The composition of claim 4 comprising an adjuvant.

8. The composition of claim 4, wherein the composition is formulated for intranasal, intraocular, oral, mucosal, intramuscular, subcutaneous, or in ovo administration.

9. The composition of claim 4, wherein the composition is formulated for spraying or aerosolizing.

10. A vaccine comprising the isolated Mycoplasma gallisepticum of claim 1.

11. The vaccine of claim 10, wherein the vaccine reduces one or more of the clinical signs induced by Mycoplasma gallisepticum infection in poultry.

12. The vaccine of claim 10, wherein the vaccine reduces the susceptibility of a birds of the order Galliformes to disease induced by Mycoplasma gallisepticum.

13. A live vaccine for birds of the order Galliformes, the vaccine comprising an amount of the K6067 Mycoplasma gallisepticum strain deposited at the ATCC under Patent Deposit Designation PTA-127168 or a progeny or derivative thereof, or the K4110 Mycoplasma gallisepticum strain deposited at the ATCC under Patent Designation PTA-127282, and progeny and derivatives thereof sufficient to protect the birds from disease induced by Mycoplasma gallisepticum, and a pharmaceutically acceptable carrier.

14. The vaccine of claim 10, wherein the isolated Mycoplasma gallisepticum, composition, or vaccine is lyophilized, freeze dried, frozen, or an effervescent tablet.

15. A kit comprising the isolated Mycoplasma gallisepticum of claim 1 and printed instructions, wherein the contents of the kit are contained within packaging material.

16. A method of producing an immune response to Mycoplasma gallisepticum in a bird, the method comprising administering the isolated Mycoplasma gallisepticum of claim 1 to the bird.

17. A method for reducing susceptibility of a bird against disease induced by Mycoplasma gallisepticum, the method comprising administering the isolated Mycoplasma gallisepticum of claim 1 to the bird.

18. A method for protecting a bird against Mycoplasma gallisepticum infection, the method comprising administering the isolated Mycoplasma gallisepticum of claim 1 to the bird.

19. A method of reducing one or more clinical signs induced by a Mycoplasma gallisepticum infection in a bird, the method comprising administering an effective amount of the isolated Mycoplasma gallisepticum of claim 1 to the bird.

20. The method of claim 16, wherein
administration is intranasal, intraocular, oral, mucosal, intramuscular, subcutaneous, or in ovo or by eye drop, by aerosol, or by drinking water.

21. (canceled)

22. The method of claim 16, wherein the bird comprises a bird of the order Galliformes.

23. The method of claim 16, wherein the bird comprises a chicken or a turkey.
